(19) United States
(12) Patent Application Publication
(10) Pub. No.: US 2005/0281775 A1
(43) Pub. Date: Dec. 22, 2005

(54) Mucoadhesive and bioadhesive polymers

(76) Inventors: Stephen Dennis Carrington, Roundwood (IE); David Haddleton, Warwickshire (GB); David James Brayden, Glenageary (IE)

Correspondence Address: Craig G. Cochenour, Esq.;Buchanan Ingersoll PC, One Oxford Centre, 20th Floor, 301 Grant Street, Pittsburgh, PA, 15219, US

(21) Appl. No.: 10/869,226

(22) Filed: Jun. 16, 2004

(51) Int. Cl. A61K 7/06; A61K 7/11

(52) U.S. Cl. 424/70.16

(57) ABSTRACT

This invention provides a method of augmenting an epithelial mucosal barrier, by contacting the barrier with a topical composition. This invention also provides the topical composition.
The topical composition comprises, dispersed or dissolved in a topically acceptable vehicle, at least one polymer containing either a repeat unit of structure 1 or a repeat unit of structure 2, or a mixture 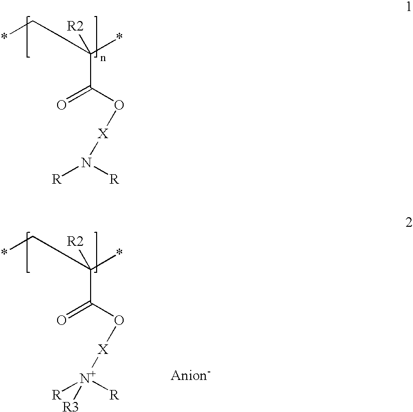 This method and composition are useful in improving mucosal barrier function by, for example, topical application to an exposed or injured epithelial surface or by coating a compromised mucosal barrier in, for example, inflammatory bowel disease.

### BACKGROUND

Mucosal barrier function may be compromised where the (non-stirred) secreted mucus gel that normally surmounts the mucosal barrier is compromised (e.g. inflammatory bowel disease). In such cases, substances that are normally restricted to the supramucosal environment (e.g. microbes, microbial toxins, proinflammatory substances, or high molecular weight digestion products in the gut) may gain access to the mucosal epithelium, or pass into or through it, thereby causing pathological effects.

Known mucoadhesives and bioadhesives include various chemical derivatives of chitosan, Carbopol (Trade Mark) and polycarbophil. Chitosan derivatives are known to open epithelial tight junctions, prevent intestinal ulceration, retain drugs in open wounds, increase ocular-surface residence, and have vaccine adjuvant activity. However, chitosan has poor solubility in non-acid pH. A derivative, n-trimethyl chitosan, has better solubility at a range of pH values. However, by opening tight junctions, chitosan may permit the entry of pathogens and this may be a disadvantage by comparison with polymers that augment epithelial barrier function.

It is an object of the invention to identify polymers associated with augmenting epithelial barrier function.

The present invention addresses these problems by providing exogenous synthetic polymers based on, or similar to, poly (methacrylate)s that adhere to cells (bioadhesion) and/or mucosal gels (mucoadhesion). The polymers used herein augment the barrier afforded by the residual mucus surmounting the epithelial surface. This may reduce the access of harmful substances in the supramucosal environment to the epithelium and associated tissues. The polymers used herein appear to increase the barrier function by reducing paracellular flux of small polar molecules through epithelial tight junctions. They may also integrate into mucus; thereby making it more viscous; less likely to turn over; and/or less porous, and thus less able to transmit substances by diffusion. For example, the polymers reduce access of a known bacterial toxin to the epithelium: probably through a reduction in the pore-size of the mucus gel.

The problem to be addressed by the current invention is to identify polymers that have a high capacity to adhere to cells or mucus gels. The polymers used herein have the following characteristics. They:—
display a high level of bio- and mucoadhesion
are readily synthesised in bulk by reproducible methods
are soluble in aqueous solutions (physiological pH range)
are non-toxic to the underlying epithelium
promote the improvement of barrier function
have inherent antibiotic capability
are characterised with respect to their effect on the permeability of whole-mucosal layers (ex vivo) and isolated epithelial sheets.

We have synthesised candidate polymers, using the process of living radical polymerisation taught in (WO 0194424, incorporated herein by reference), and by other methods such as other living radical polymerisation techniques such as ATRP (Matyjaszewski, K.; Xia, J. Chem. Rev. 2001, 101, 2921 and Kamigaito, M.; Ando, T.; Sawamoto, M. Chem. Rev. 2001, 101, 3689); nitroxide (Hawker, C.; Bosman, A. W.; Harth, E. Chem. Rev. 2001, 101, 3661) and RAFT (Chiefari, J.; Chong, Y. K.; Ercole, F.; Krstina, J.; Jeffery, J.; Le, T. P. T.; Mayadunne, R. T. A.; Meijs, G. F.; Moad, C. L.; Moad, G.; Rizzardo, E.; Thang, S. H. Macromolecules 1998, 31, 5559-5562). We have evaluated the performance of these polymers in vitro, using whole rat intestinal epithelial tissues, and human intestinal epithelial cells lines (including a line that produces a mucus gel layer). These have been compared with trimethylated chitosan (TMC) as a current industry standard for high levels of mucoadhesion. The polymers were selected based on their solubility and ease of synthesis. We evaluated their mucoadhesivity, bioadhesivity, toxicity, and their effects on epithelial and mucosal permeability in vitro.

The following account describes the design characteristics for polymers that we have shown to have a high capacity to adhere to cells and/or mucus, and which have, surprisingly, a low cytotoxicity. We have also shown that some of them, surprisingly, enhance the barrier of mucosal layers to standard flux markers and substantially reduce cytotoxicity normally caused by a microbial toxin (Example 4).

### SUMMARY OF THE INVENTION

According to a first aspect of the invention, there is provided a method of augmenting an epithelial mucosal barrier, the method comprising contacting the epithelial mucosal barrier with a topical composition comprising, dispersed in a topically acceptable vehicle, preferably dissolved in the topically acceptable vehicle, at least one polymer either
a. containing a repeat unit of structure 1 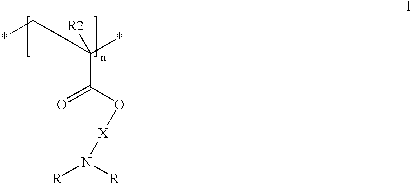
   in which each R is independently H, a lower C1-12 straight or branched alkyl such as Me, Et, But, a lower C2-12 alkenyl or a lower C2-12 alkynyl or a substituted or unsubstituted aryl or both R together form a cyclic or a heterocyclic ring; R2 is H or a lower C1-12 straight or branched alkyl such as Me, Et, but preferably Me or H; X is a bivalent radical selected from a C1-5 alkylene, C2-5 alkenylene or C2-5 alkynylene radical which may be interrupted by a heterocyclic atom such as oxygen; and n is between 2 and 2000, optionally between 5 and 1000, preferably between 5 and 200; or
b. containing a repeat unit having the structure 2 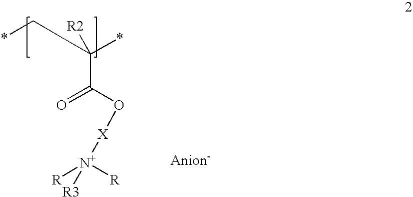
   in which R, X, R2 and n are as defined above; and R3 is H, lower C1-12 alkyl, lower C2-12 alkenyl or lower C2-12 alkynyl or a substituted or unsubstituted aryl; and anion is a monovalent anion, preferably a halide such as Cl—, I—, Br—;
or a mixture of repeat units having structures 1 and 2.

According to a second aspect of the invention, there is provided a topical composition for topical application to a mucosal surface, the topical composition comprising at least one polymer containing either a repeat unit of structure 1 or of structure 2, or a mixture of repeat units of structures 1 and 2, dispersed in a topically acceptable vehicle.

By mucosal surface is meant any surface selected from the group consisting of ocular, respiratory, alimentary, reproductive, urinary and intact as well as exposed and/or injured epithelial surfaces. The term “respiratory” embraces nasal, laryngeal, tracheal and pulmonary surfaces. The term “alimentary” embraces the gastrointestinal tract including buccal, oesophageal, gastric, small and large intestinal and rectal surfaces. The term “urinary” embraces any mucosal surface that is normally exposed to urine, such as the bladder. The term “reproductive” embraces salpingial, uterine, vaginal and vestibular surfaces, together with homologous surfaces in the male reproductive tract such as the ductus deferens surfaces. The term “injured, epithelial surfaces” comprises any epithelial surface in which the epithelium, for whatever reason, is no longer intact. This can happen where, for example, the skin is cut or is ulcerated, bumt, chemically damaged or subjected to any insult that affects its integrity and viability.

Polymers based on DMAEMA, either unquaternised or quaternised, form a preferred group of polymers useful in the invention but it is not intended that the invention be so limited.

The polymers mentioned above optionally have a polydispersity index between 1.00 and 5.00 but preferably between 1.00 and 2.50 and more preferably between 1.00 and 1.50. Polydispersity is a measure of the weight distribution of the polymer and is calculated by dividing the weight average molecular weight by the number average molecular weight. It will be appreciated that a polydispersity index as close to 1.00 as possible is preferred for use in the present invention.

The polymers useful in the present invention optionally have a weight average molecular weight of less than about 500,000, optionally less than about 150,000 or less than about 100,000, preferably about less than about 75,000, more preferably less than about 50,000. Without being bound by theory, it is believed that a lower weight average molecular weight is desirable, for use within the method and composition of the present invention.

The polymers useful in the present invention preferably have a number average molecular weight of less than about 25,000, preferably less than about 16,500. Without being bound by theory, it is believed that a lower number average molecular weight is desirable for the method and composition of the present invention.

Since, for each polymer, the polydispersity index is obtained by dividing the weight average molecular weight by the number average molecular weight, it will be appreciated that both these factors are inter-related and are reflected, to some extent, in the polydispersity index.

The polymers mentioned above are obtainable by radical, anionic polymerisation or group transfer; preferably by living radical polymerisation, including transition metal mediated (ATRP—Matyjaszewski, K.; Xia, J. Chem. Rev. 2001, 101, 2921 and Kamigaito, M.; Ando, T.; Sawamoto, M. Chem. Rev. 2001, 101, 3689), nitroxide mediated (Hawker, C.; Bosman, A. W.; Harth, E. Chem. Rev. 2001, 101, 3661), RAFT (Chiefari, J.; Chong, Y. K.; Ercole, F.; Krstina, J.; Jeffery, J.; Le, T. P. T.; Mayadunne, R. T. A.; Meijs, G. F.; Moad, C. L.; Moad, G.; Rizzardo, E.; Thang, S. H. Macromolecules 1998, 31, 5559-5562); more preferably by transition metal mediated polymerisation as described by U.S. Pat. No. 6,310,149. The reaction may take place with or without the presence of a solvent. Suitable solvents in which the catalyst, monomer and polymer product are sufficiently soluble for reactions to occur, include water, protic and non-protic solvents, including propionitrile, hexane, heptane, dimethoxyethane, diethoxyethane, tetrahydrofuran, ethylacetate, diethylether, N,N-dimethylformamide, anisole, acetonitrile, diphenylether, methylisobutyrate, butan-2-one, toluene and xylene. Preferred solvents are xylene and toluene, preferably the solvents are used at at least 1% by weight, more preferaby at least 10% by weight. The polymers can be prepared either by conventional processes or by the process of WOO 194424.

A suitable initiator compound comprises a hydrolytically cleavable bond with a halogen atom—“hydrolytically cleavable” means a bond which breaks without integral charge formation on either atom by homolytic fission. The initiator compound is preferably selected from 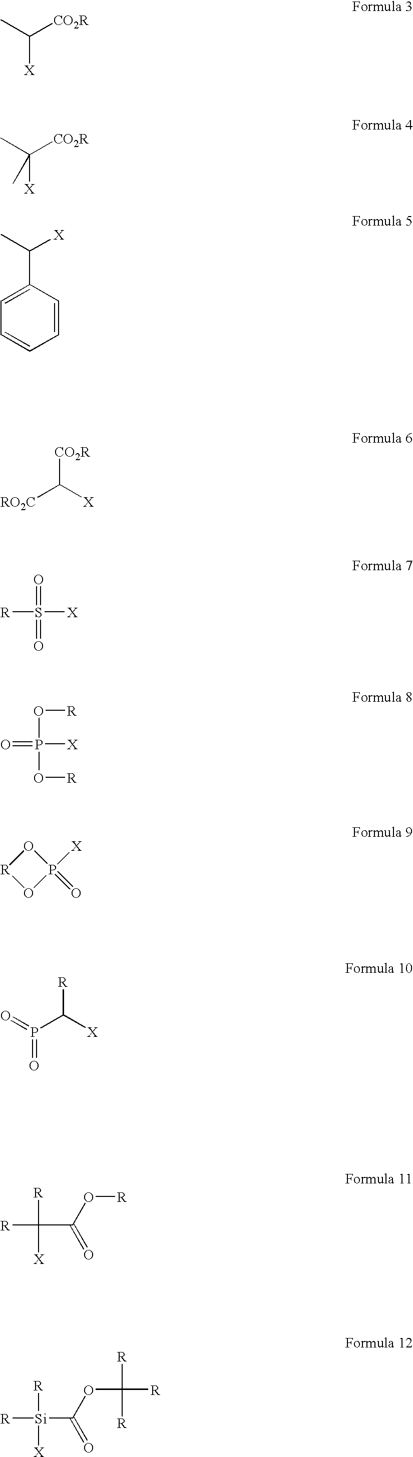
where R is independently selected from straight, branched or cyclic alkyl, hydrogen, substituted alkyl, hydroxyalkyl, carboxyalkyl or substituted benzyl. Preferably the or each alkyl, hydroxyalkyl or carboxyalkyl contains 1 to 20, especially 1 to 5 carbon atoms. X is a halide, especially I, Br, F or Cl. The initiator compound may especially be selected from Formulae 13 to 23: 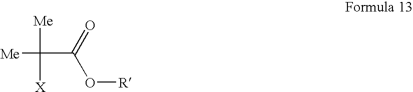
where:
X═Br, I or Cl, preferably Br
   R′═—H,
   —(CH₂)pR″ (where m is a whole number, preferably p=1 to 20, more preferably 1 to 10, most preferably 1 to 5, R″═H, OH, COOH, halide. NH₂, SO₃, COX—where X is Br, I or CL) or: 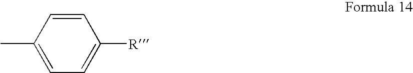
   R¹¹¹═—COOH, —COX (where X is Br, I, F or Cl), —OH, —NH₂ or —SO₃H, especially 2-hydroxyethyl-2′-methyl-2′bromo-propionate. 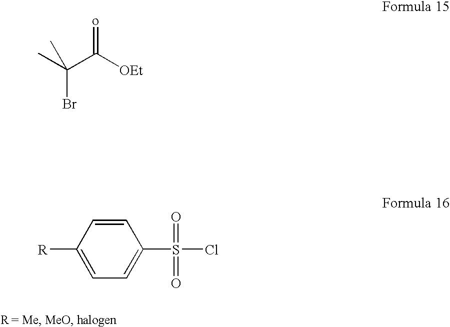
   Especially preferred examples of Formula 16 are: 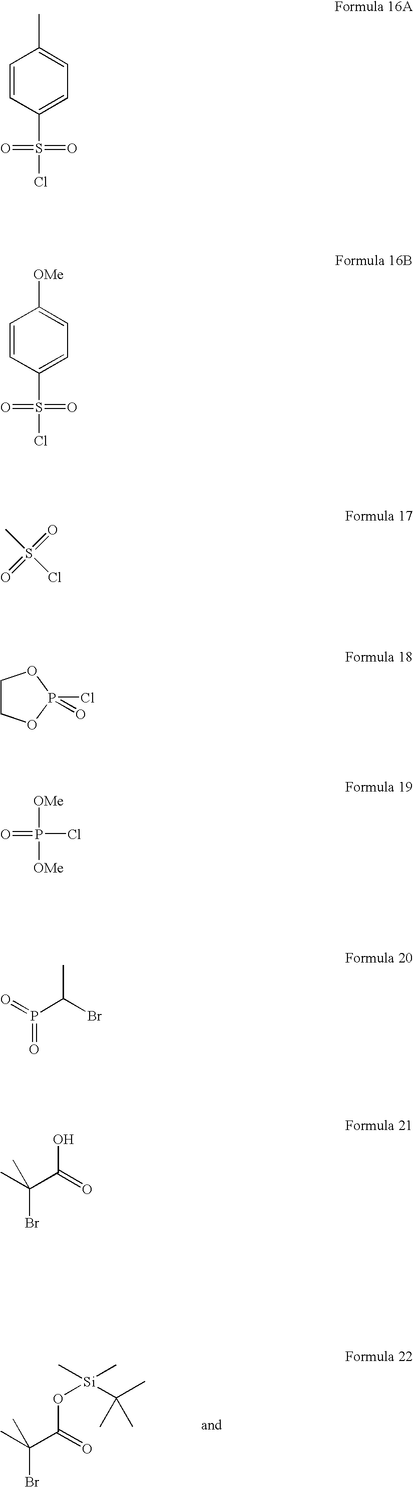 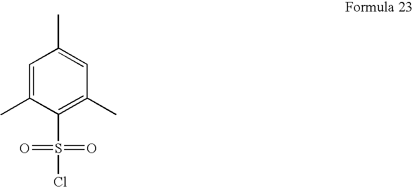
   The careful selection of functional alkyl halides allows the production of terminally functionalised polymers. For example, the selection of a hydroxy containing alkyl bromide allows the production of α-hydroxy terminal polymers. This can be achieved without the need of protecting group chemistry.

The broadest definition of a polymer useful in the methods and compositions of the present invention include, of course, polymers containing monomers other than of repeating unit structures 1 and 2. Thus, included within polymers useful in the methods and compositions of the present invention are:—
c. Statistical copolymers (i.e. copolymers in which the repeat units are randomly distributed) of the repeat units shown in structures 1 and/or 2 above with any other olefinically unsaturated monomer such as, but not limited to a methacrylic, an acrylate, a styrene, methacrylonitrile or a diene such as butadiene. Examples of olefinically unsaturated monomers that may be polymerised include methyl methacrylate, ethyl methacrylate, propyl methacrylate (all isomers), butyl methacrylate (all isomers), and other alkyl methacrylates; corresponding acrylates; also functionalised methacrylates and acrylates including glycidyl methacrylate, trimethoxysilyl propyl methacrylate, allyl methacrylate, hydroxyethyl methacrylate, hydroxypropyl methacrylate, dialkylaminoalkyl methacrylates; fluoroalkyl (meth)acrylates; methacrylic acid, acrylic acid; fumaric acid (and esters), itaconic acid (and esters), maleic anhydride; styrene, α-methyl styrene; vinyl halides such as vinyl chloride and vinyl fluoride; acrylonitrile, methacrylonitrile; vinylidene halides of formula CH₂═C(Hal)₂ where each halogen is independently Cl or F; optionally substituted butadienes of the formula CH₂═C(R¹⁵) C(R¹⁵)═CH₂ where R¹⁵ is independently H, C1 to C10 alkyl, Cl or F; sulphonic acids or derivatives thereof of formula CH₂═CHSO₂OM wherein M is Na, K, Li, N(R¹⁶)₄ where each R16 is independently H or Cl iSO₂OZ and Z is H, Li, Na, K or N(R¹⁶)₄; acrylamide or derivatives thereof of formula CH₂═CHCON(R¹⁶)₂ and methacrylamide or derivative thereof of formula CH₂═C(CH₃)CON(R16)₂. Mixtures of such monomers may be used. Preferably, the monomers are commercially available and may comprise a free-radial inhibitor such as 2,5-di-tert-butyl-4-methylphenol or methoxyphenol.
d. Block copolymers (ie where copolymers in the repeat units are arranged in blocks) (obtainable by sequential addition of monomers) of structure 3 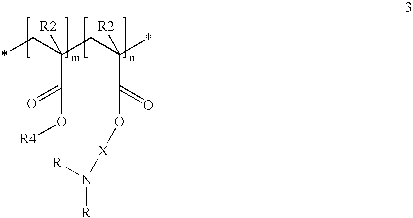
   having at least one repeat unit as defined in structure 1 above and having at least one other repeat unit in which R, n, R2 and X are as defined above; m is between 2 and 2000, optionally between 5 and 1000, preferably between 5 and 200; R4 is H, or a lower C1-12 alkyl such as Me, Et, But, lower C2-12 alkenyl or lower C2-12 alkynyl; and
e. Block copolymers of the structure 4; 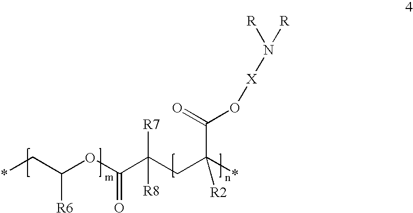
   having at least one repeat unit as defined in structure 1 above and having at least one repeat unit in which each of R6, R7 and R8, which can be the same or different, is H or Me; m is between 2 and 1000, preferably between 5 and 1000, optionally between 5 and 200; and n is between 2 and 2000, optionally between 5 and 1000, preferably between 5 and 200.

The polymers useful in the method of the present invention also include polymers of options c to e above, in which repeat unit structure 1 is partially or fully replaced with repeat unit structure 2, as well as, tri-blocks.

The polymerisation process yields a polymer that is aqueous soluble at pH 6-8.5, optionally 7-8, preferably about 7.4.

A variant of these polymers utilise multiple initiators to generate polymers configured as multi-arm ‘stars’, which star polymers are disclosed, for example, in Angot, S.; Murthy, K. S.; Taton, D.; Gnanou, Y. Macromolecules 1998, 31, 7218-7225 and Haddleton, D. M.; Edmonds, R.; Heming, A. M.; Kelly, E. J.; Kukulj, D. N. J. Chem. 1999, 23, 477-479.

Polymers containing repeat unit structure 2, and containing tertiary amine group, appear to be preferred for mucoadhesion, since these appear to be the main determinant for this characteristic.

The methods of the present invention are useful in augmenting the mucosal barrier by, for example,
Enhancement of the barrier afforded by mucosal surfaces (e.g. through reduction in gel pore size, or closure of epithelial tight junctions) (see, for example, Examples 2 and 4)
Protection of endogenous mucus gels from degradation
Reduction/prevention of microbial access to epithelial surfaces (Wu et al., 2004)
   Inhibition of microbial colonisation of epithelial surfaces
   Inhibition of microbial viability and multiplication at epithelial surfaces
Reduction/prevention of microbial toxin access to epithelial surfaces (see Example 4)
   Utility

Polymers based on methacrylate are known. The method of the present invention, however, discloses some unique beneficial properties of a series of hydrophilic variations based on iterations of a monomer of methacrylate and monomers similar thereto. Using monolayers and isolated rodent non-everted sacs, we have shown a rank order suggesting that certain forms of pDMAEMA are more mucoadhesive and less bioadhesive, whilst other forms are more muco- and more bio-adhesive, than a hydrophilic chitosan derivative. In the range of pDMAEMA derivatives tested, some displayed a strong bias to mucoadhesivity over bioadhesivity. Others were similarly mucoadhesive and bioadhesive. We have also shown, in contrast to chitosan, that barrier function was improved and that the polymer had an ability to prevent the damaging actions of components of a bacterial exotoxin, cholera toxin. These polymers have commercial value for the following reasons:
1. Easy cheap synthesis and characterisation in scale-up.
2. Potential to improve barrier function, for example, by applying, topically, to an exposed wound.
3. Potential to improve barrier function, for example, by coating the inflamed gut. The polymers alone would therefore be useful for the treatment of inflammatory bowel disease.
4. Inherent antibiotic action, again by augmenting the barrier function.
5. The ability to select polymers that are either similarly mucoadhesive and bioadhesive or, alternatively, more mucoadhesive than bioadhesive.
   Methods for Preparing Polymers Useful in the Present Invention
   Synthesis of Hostasol Monomer and Initiator

### Thioxantheno[2,1,9-dej]isochromene-1,3-dione

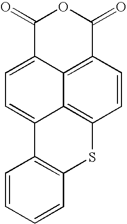 A suspension of crude anhydride (100 g) in N-methylpyrrolidone (750 g) was heated at 130° C. for one hour. The mixture was cooled to 85° C. and filtered whilst hot to give a crude syrupy orange solid (Compound still wet, contains N-methylpyrrolidone, can't evaporate solvent, boiling point around 230° C.). Diethyl ether was added to a batch of this material (˜80 g) and shaken vigorously and the orange solid filtered. The solid was washed with diethyl ether (3×200 mL), suction dried, and dried in a vacuum oven at 50° C. overnight to give orange coloured product (26.08 g). mp 315-317° C.; ¹H NMR (400 MHz, CDCl₃) δ 7.48-7.60 (m, 2H), 7.59-7.65 (m, 1H), 7.78 (d, 1H, J=8.0 Hz), 8.30 (d, 1H, J=8.0 Hz), 8.45-8.55 (m, 3H);

### 2-(8-hydroxy-3,6-dioxaoctyl)thioxantheno[2,1,9-dej]isoqiunoline-1,3-dione

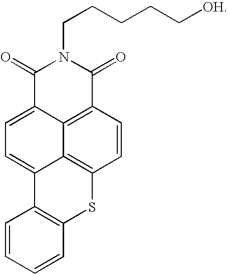 Hostasol anhydride (10 g, 32.86 mmol) was suspended in anhydrous DMF (60 mL) under nitrogen at room temperature. 5-Aminopentanol (20.34 g, 197.15 mmol) and p-toluenesulfonic acid monohydrate (PTSA) (0.375 g, 1.971 mmol) were added. The reaction mixture was stirred at 130° C. under nitrogen and followed by TLC (toluene-methanol, 4:1 (v/v), R_{f} 0.45, intense orange spot). A further portion of pTSA (0.375 g, 1.971 mmol) and 5-amnopentanol (1 g, 9.69 mmol) were added at t=3 hours. After 6.5 hours, the reaction mixture was allowed to cool to 50° C. and methanol was added (30 mL). The resulting bright orange solid was filtered and washed with cold methanol (3×40 mL). The solid was dried under vacuum at 80° C. to give alcohol (11.83 g, 92%) as a bright orange solid. mp 150-152° C.; ¹H NMR (400 MHz, d⁶-DMSO) δ1.30-1.38 (m, 2H, H-3), 1.43-1.51 (m, 2H, H-2), 1.52-1.62 (m, 2H, H-4), 3.38-3.44 (m, 2H, H-1), 3.87 (t, 2H, J=7.5 Hz, H-5), 4.37 (t, 1H, J=5.1 Hz, OH), 7.27-7.33 (m, 3H), 7.34-7.39 (m, 1H), 7.95 (d, 1H, J=7.8 Hz), 8.00 (d, 1H, J=8.5 Hz), 8.05-8.10 (m, 2H); ¹³C NMR (100 MHz, d⁶-DMSO) δ23.1 (C-3), 27.2 (C-4), 32.2 (C-2), 39.6 (C-5), 60.5 (C-1), 117.1, 119.4, 120.1, 120.2, 123.9, 126.1 (x2), 126.8, 127.6, 129.0, 129.7, 130.1, 130.3, 131.5, 135.3, 139.1, 162.0 (C═O), 162.5 (C═O); HRMS (EI+) calcd for C₂₃H₁₉NO₃S+: 389.1086, found: 389.1079.

### 2-(8-methacryloyloyx-3,6-dioxaoctyl)thioxantheno[2,1,9-dej]isoquinoline-1,3-dione

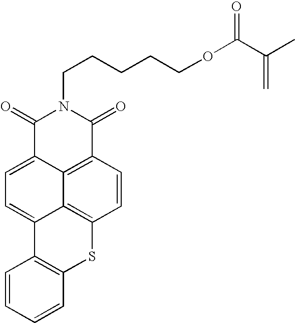 Anhydrous chloroform (80 mL) was added to a 250 mL RBF flask containing alcohol (2 g, 5.14 mmol) under an atmosphere of nitrogen at room temperature. Triethylamine (3.6 mL, 26.09 mmol) was added and the reaction mixture cooled to 0° C. (ice-bath). A solution of methacroyl chloride (2.4 mL, 25.01 mmol) in anhydrous chloroform (20 mL) was added dropwise over a period of 1 hour. The reaction mixture was then left to stir at room temperature overnight and was followed by TLC (toluene-ethyl acetate, 5:1 (v/v). After 18 hours, the reaction mixture was cooled to 0° C. (ice-bath) and methanol (10 mL) was added to destroy excess methacroyl chloride and the mixture stirred for 1 hour at room temperature. The reaction mixture was washed successively with saturated aqueous sodium chloride solution (3×100 mL), saturated aqueous sodium hydrogen carbonate (100 mL), saturated aqueous sodium chloride solution (100 mL), dried over MgSO₄, filtered and evaporated under reduced pressure to give an orange solid which was purified by silica chromatography (toluene-ethyl acetate, 5:1 (v/v)) and co-evaporating with methanol to give monomer (2.07 g, 86%) as an orange solid (NB on evaporating toluene, obtain a brown syrup. Co-evaporating with methanol gives an orange solid, CAUTION, solid material bumps a lot as solvent evaporates) mp 122-124° C.; ¹H NMR (400 MHz, CDCl₃) δ1.45-1.58 (m, 2H, H-3), 1.70-1.83 (m, 4H, H-2, 4), 1.92 (s, 3H, CH₃), 4.11-4.19 (m, 4H, H-1,5), 5.50-5.53 (m, 1H, H-7a), 6.06-6.10 (m, 1H, H-7b), 7.26-7.37 (m, 4H, H_{d,e,f,g}), 7.98 (d, 1H, J=8.3 Hz, H_{b}), 8.01-8.07 (m, 1H, H_{c}), 8.26 (d, 1H, J=8.0, Hₕ), 8.43 (d, 1H, J=8.3 Hz, Hₐ); ¹³C NMR (100 MHz, CDCl₃) δ18.3 (CH₃), 23.6 (C-3), 27.6 (C-4), 28.4 (C-2), 40.1 (C-5), 64.6 (C-1), 117.9, 118.9 (C_{b}), 120.2, 121.0, 125.1, 125.2 (C-7), 125.9 (C_{c}), 126.3, 127.5, 129.9 (C_{e,f,g}), 127.7, 130.1, 130.5 (Cₕ), 131.5, 132.3 (Cₐ), 136.4 (C-6), 136.5, 140.3, 163.2 (C═O, ring), 163.6 (C═O, ring), 167.5 (C═O); HRMS (EI⁺) calcd for C₂₇H₂₃NO₄S+: 457.1348, found: 457.1362.

### 2-(8-(2-bromodimethyl)-3,6-dioxa-octyl)-thioxantheno[2,1,9-dej]isoquinoline-1,3-dione (Hostasol Initiator)

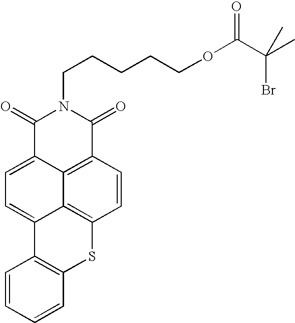 Anhydrous chloroform (80 mL) was added to 250 mL RBF containing hostasol alcohol (2 g, 5.135 mmol) under an atmosphere of nitrogen at room temperature. Triethylamine (3.6 mL) was added and the reaction mixture cooled to 0° C. (ice-bath). A solution of 2-bromoisobutyryl bromide (3.1 mL) in anhydrous chloroform (20 mL) was added dropwise over a period of 1 hour. The reaction mixture was left to stir overnight at room temperature and was followed by TLC (toluene-ethyl acetate, 5:1 (v/v), R_{f} 0.52). After 18 hours, the reaction mixture was cooled to 0° C. (ice-bath), methanol (10 mL) added, and the mixture stirred for 1 hour at room temperature. The reaction mixture was washed successively with saturated aqueous sodium chloride solution (3×100 mL), saturated aqueous sodium hydrogen carbonate (100 mL), saturated aqueous sodium chloride solution (100 mL), dried over MgSO₄, filtered and evaporated under reduced pressure to give a bright orange solid. The solid was dissolved in chloroform (40 mL) and hexane (160 mL) was added. Crystals start to form after a little while at room temperature and completed in a fridge overnight to give initiator (2.44 g, 89%) as bright orange crystals. mp 143-144° C.; ¹H NMR (400 MHz, CDCl₃) δ1.45-1.58 (m, 2H, H-3), 1.67-1.85 (m, 4H, H-2,4), 1.91 (s, 6H, 2×CH₃), 4.10-4.20 (m, 4H, H-1,5), 7.26-7.38 (m, 4H, H_{d,e,f,g}), 8.00 (d, 1H, J=8.5 Hz, H_{b}), 8.03-8.08 (m, 1H, H_{c}), 8.27 (d, 1H, J=7.8 Hz, Hₕ), 8.45 (d, 1H, J=8.0 Hz, Hₐ); ¹³C NMR (100 MHz, CDCl₃) δ23.4 (C-3), 27.5 (C-4), 28.1 (C-2), 30.7 (CH₃), 40.0 (C-5), 65.9 (C-1), 117.9, 119.0 (C_{b}), 120.2 (C_{g}), 121.0, 125.2, 125.9 (H_{c}), 126.3, 127.5, 129.9 (C_{e,f,g}), 127.8, 130.1, 130.6 (Ch), 131.5, 132.3 (Hₐ), 136.4, 140.3, 163.2 (C═O, ring), 163.7 (C═O, ring), 171.673 (C═O); HRMS (EI⁺) calcd for C₂₇H₂₄BrNO₄S+: 538.0631, found: 538.0628.
Polymerisation of Hostasol Initiator and DMAEMA to Form Polymer “a” 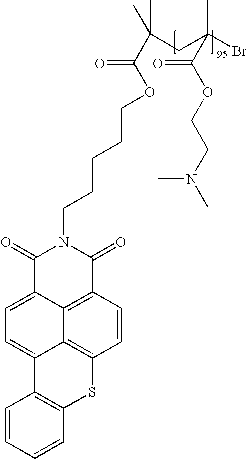
Cu^{(I)}Br (426 mg) and hostasol initiator (1.6 g) were weighed out into a schlenk tube and sealed with a subaseal. (Dimethylamino)ethyl methacrylate (40 mL, filtered through alumina and degassed) and toluene (80 mL) were added. The mixture was frozen in liquid nitrogen and degassed via four freeze-pump-thaw cycles. The reaction mixture, under an atmosphere of nitrogen, was placed in a oil bath at 90° C. and stirred for 5 minutes. Propyl pyridine-2-yl methyleneamine (propyl ligand) (0.93 mL) was added to initiate polymerisation. The polymerisation mixture was heated to 90° C. under an atmosphere of nitrogen. Reaction aliquots (0.4 mL) were removed at times, 30, 60, 90, 300 and 360 minutes. Polymerisation was followed by ¹H-NMR.

After 360 minutes, the reaction mixture was removed from the oil bath, diluted with toluene (˜50 mL) and filtered through a column of basic alumina to remove the copper catalyst. The column was washed with two column volumes of toluene and toluene/ethyl acetate (1:1, v/v), and the filtrate evaporated under reduced pressure. The polymer was dissolved in chloroform (˜120 mL) and added dropwise to a beaker of light petroleum with vigorous stirring to precipitate the polymer. The solvent was decanted off and the polymer reconstituted in chloroform and evaporated under reduced pressure to give Hostasol pDMAEMA (30 g, Mn 15000) as a brown crystalline solid.

Synthesis of High Molecular Weight pDMAEMA Polymer
1% AIBN Catalysed Free-Radical Polymerisation of DMAEMA+1% Hostasol Monomer—Polymer “B” 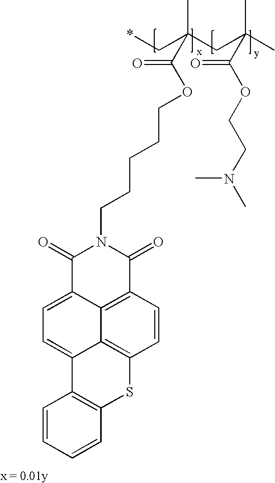
Hostasol monomer (0.1 g, 0.219 mmol (1% by wt)) (prepared as described above) and AIBN (0.1 g, 1% by wt) were weighed out into a Schlenk tube and the tube sealed with a subaseal. Dimethylaminoethyl methacrylate (10.7 mL, 10 g, 63.61 mmol) and toluene (35 mL) were added by syringe and the reaction mixture then degassed by four freeze-pump-thaw cycles. The reaction mixture was heated at 60° C. (oil-bath) under an atmosphere of nitrogen for 48 hours. After 48 hours, the fluorescent viscous polymer solution was evaporated under reduced pressure and dried under high vacuum to give polymer (12.18 g) as a brown orange crystalline solid. This polymer has a Mn 22800; Mw 103600 and a polydispersity index (Pdi) 4.54.
General Procedure of Quaternisation of Hostasol pDMAEMA (High MW) Polymers—Polymer “C”

Polymer (1 g) was dissolved in 20 mL of 1M HCl (aq) with stirring. The orange coloured solution was frozen in liquid nitrogen and freeze-dried to give quaternised high molecular weight (MW) pDMAEMA polymers as brown/orange solids.

Quaternisation of Hostasol pDMAEMA with MeI 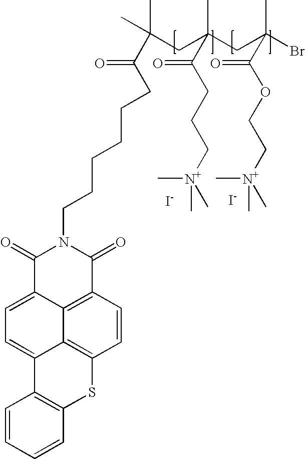
Hostasol pDMAEMA was reacted with methyl iodide to give polymers of the desired quaternisation, for example, 33%, 66% and 100% quaternisation.
General Procedure

Hostasol pDMAEMA (100 mg) was dissolved in anhydrous THF (7 mL) at room temperature under an atmosphere of nitrogen. Methyl iodide (12.5 μl, 0.201 mmol; 25.1 μl, 0.403 mmol; 38 μl, 0.610 mmol; for quaternising to 33%, 66% and 100% respectively) was added and the mixture stirred at room temperature for 7 hours. Precipitation of the polymer starts to occur after ˜20-30 minutes. The reaction mixture was diluted with hexane and the quaternised polymer was filtered.
Quaternisation of Hostasol pDMAEMA with HCl to form Polymer “N” 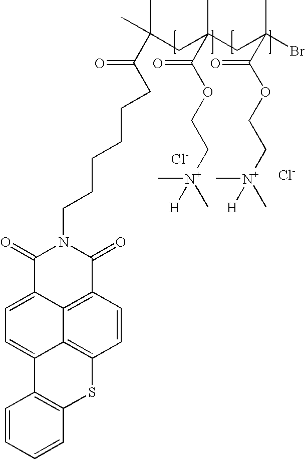
Hostasol pDMAEMA (10 g) was dissolved in 100 mL of 1M HCl (aq) with stirring. The orange coloured solution was frozen in liquid nitrogen and freeze-dried to give quaternised Hostasol pDMAEMA (11.4 g) as an orange powder.
Trimethylated Chitosan Chloride—Comparative Polymer “Z” 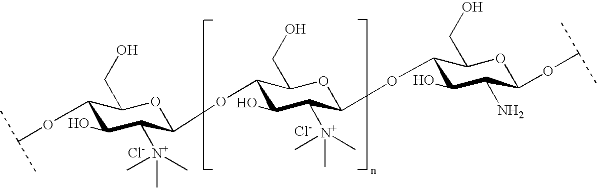
1-Methyl-2-pyrolidinone (80 mL) was added to a flask containing chitosan (2 g) (low viscous, Fluka) and sodium iodide (4.8 g). The mixture was heated at 60° C. to dissolve the chitosan. Sodium hydroxide (aq) (11 mL, 15% w/v) and iodomethane (11 mL) were added, a condenser attached and the reaction mixture stirred at 60° C. for one hour. The mixture was removed from the oil bath and ethanol (200 mL) was added to precipitate the derivatised chitosan. The suspension was transferred to suitable containers and centrifuged for 10 minutes at 4000 g. The supernatant was decanted off. The solid was sequentially washed thoroughly with ethanol (×2) and diethyl ether and product isolated by centrifugation. The isolated solid was transferred to a round bottomed flask to which 1-methyl-2-pyrolidinone (80 mL) was added. The suspension was heated to 60° C. to dissolve the solid and then sodium iodide (4.8 g), sodium hydroxide (aq) (11 mL, 15% w/v) and iodomethane (11 mL) were added. The mixture was heated with an attached condenser at 60° C. for 30 minutes. An additional iodomethane (2 mL) and sodium hydroxide pellets (2 g) were added and stirring continued for 1 hour. The product was washed and isolated as described above. The isolated product was dissolved in 10% (w/v) aqueous sodium chloride (80 mL) and stirred for 30 minutes. The product was precipitated with ethanol and isolated by centrifugation to yield trimethylated chitosan as the chloride salt.
Coumarin-Labelled Trimethylated Chitosan Chloride—Comparative Polymer “Z” 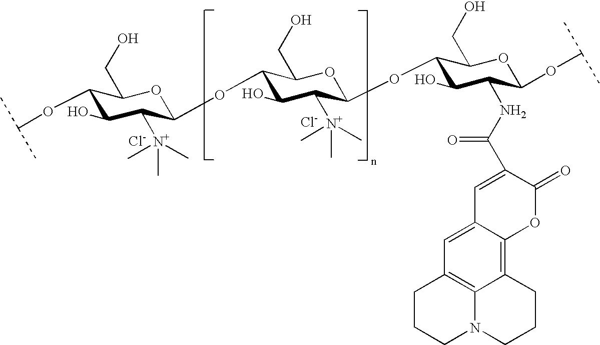
A mixture of N-trimethylated chitosan chloride salt (200 mg) in water (20 mL) was stirred at room temperature for 25 minutes to allow the chitosan to dissolve. Coumarin 343 (2 mg, 1% w/w) was added followed by DMAP (1 crystal). EDCI (CAS No. 25952-53-8) (6.7 mg) was added and the mixture was stirred at room temperature (Coumarin, insoluble to start with, dissolves as reaction proceeds). After 26 hours, the reaction mixture was freeze-dried. The solid was dissolved in 25% ethanol in water (40 mL), transferred to a dialysis bag (Molecular weight cut-off (MWC) 12-14,000) and dialysed against 25% ethanol in water (4 L). Dialysed for 7 days increasing ethanol concentration to 60% (to remove unreacted Coumarin 343), then back to 100% water. The contents of the dialysis bag were freeze-dried to yield the labelled-chitosan as a pale yellow lightweight solid.

Synthesis of Star Initiators

### 1,3,5-tri-O-isobutyryl bromide benzene (3-arm initiator)

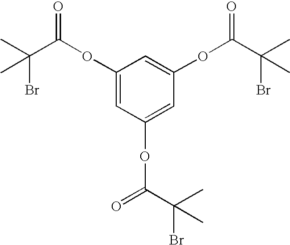 1,3,5-Trihydroxybenzene (6.31 g, 50 mmol) was dissolved in anhydrous THF (250 mL) at room temperature under an atmosphere of nitrogen. Triethylamine (23 mL, 165 mmol) was added and the mixture was cooled to 0° C. (ice bath). A solution of bromoisobutyryl bromide (20.4 mL, 165 mmol) in anhydrous THF (40 mL) was added dropwise over a period of 1 hour. A white precipitate of triethylammonium bromide forms almost immediately. After addition of bromoisobutyryl bromide, the mixture was stirred for 4 hours. The reaction mixture was filtered and the solvent evaporated under reduced pressure. The white/yellow powder was recrystallised from methanol and the product dried under vacuum to give the 3-arm initiator (25.9 g, 91%) as a white crystalline solid. mp 48-50° C.; ¹H NMR (400 MHz, CDCl₃) δ 2.04 (S, 18H, CH₃), 6.96 (s, 3H, H-1); ¹³C NMR (100 MHz, CDCl₃) δ 30.5 (CH₃), 54.8 (C-4), 112.5 (C-1), 151.3 (C-2), 169.4 (C═O);

### 1,2,3,4,6-Penta-O-isobutyryl bromide-α-D-glucose (5-arm initiator)

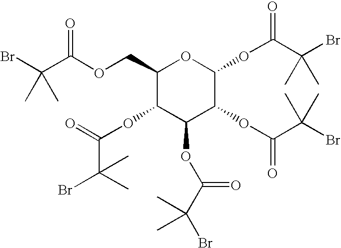 Glucose (50 g, 0.278 mol) was suspended in a mixture of anhydrous pyridine (200 mL) and anhydrous chloroform (350 mL) under an atmosphere of nitrogen at room temperature. The suspension was cooled to 0° C. (icebath) and a solution of 2-bromoisobutyryl bromide (205 mL, 1.67 mol) in anhydrous chloroform (100 mL) was added dropwise over a period of 1 hour. The reaction mixture was allowed to warm to room temperature and stirred for 4 days. The reaction mixture was diluted with chloroform (300 mL) and washed successively with ice-water (500 cm), saturated aqueous (500 mL×3), water (500 mL), dried (MgSO₄), filtered and evaporated to give a pale orange cake. Methanol (1 l) was added and the suspension stirred to break up the cake to give a fine suspension. The solid was filtered, washed with methanol (500 mL×2) and dried to give product (214.3 g, 84%) as a white powder. mp 207-208° C.; ¹H NMR (400 MHz, CDCl₃) δ1.82 (s, 3H, CH₃), 1.85 (s, 3H, CH₃), 1.86 (s, 3H, CH₃), 1.88 (x2) (s, 3H, CH₃), 1.90 (s, 3H, CH₃), 1.91 (s, 3H, CH₃), 1.94 (s, 3H, CH₃), 1.85 (s, 3H, CH₃), 2.01 (s, 3H, CH₃), 4.31-4.42 (m, 3H, H-5,6a, 6b), 5.23 (dd, 1H, J=3.8, 10.2 Hz, H-2), 5.27-5.36 (m, 1H, H-4), 5.67 (t, 1H, J=9.8 Hz, H-3), 6.39 (d, 1H, J=3.8 Hz, H-1); ¹³C NMR (100 MHz, CDCl₃) δ30.1 (CH₃), 30.2 (CH₃), 30.3 (CH₃), 30.4 (x3) (CH₃), 30.5 (CH₃), 30.6 (x2) (CH₃), 30.7 (CH₃), 54.8, 54.9, 55.0, 55.3 (x2) (C-7), 62.5 (C-6), 68.0 (C-4), 70.1 (C-5), 70.4 (C-2), 70.5 (C-3), 89.4 (C-1), 169.2 (C═O), 169.7 (C═O), 170.2 (C═O), 170.3 (C═O), 171.1 (C═O);
Synthesis of 8-Arm Initiator 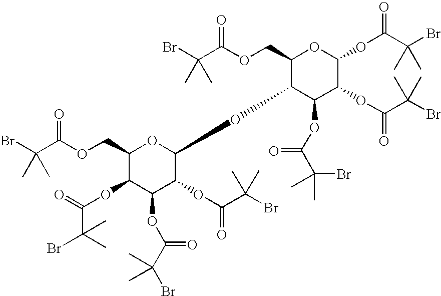
Lactose (30 g, 87.63 mmol) was suspended in a mixture of anhydrous pyridine (200 mL) and anhydrous chloroform (200 mL) under an atmosphere of nitrogen at room temperature. The suspension was cooled to 0° C. (icebath) and a solution of 2-bromoisobutyryl bromide (104 mL, 0.841 mol) in anhydrous chloroform (100 mL) was added dropwise over a period of 1 hour. The reaction mixture was allowed to warm to room temperature and stirred for 3 days. The reaction mixture was diluted with chloroform (300 mL) and washed successively with ice-water (500 cm), saturated aqueous sodium hydrogen carbonate (500 mL×3), water (500 mL), dried (MgSO₄), filtered and evaporated to give an orange transparent solid/syrup, which still contained pyridine. Ethanol (500 mL) was added to the solid and heated to boiling to break up the solid. The mixture was allowed to cool and the solid filtered, washed with light petroleum and dried to give product (128.5 g, 96%) as a white powder. mp 219-220° C.; ¹H NMR (400 MHz, CDCl₃) δ1.77 (CH₃), 1.86 (CH₃), 1.87 (CH₃), 1.88 (CH₃), 1.89 (CH₃), 1.90 (CH₃), 1.92 (CH₃), 1.93 (CH₃), 1.94 (CH₃), 1.94 (CH₃), 1.95 (CH₃), 1.96 (CH₃), 2.00 (CH₃), 2.00 (CH₃), 2.00 (CH₃), 2.02 (CH₃), 4.16-4.26 (m, 3H, H-4,5′,6a′), 4.29-4.39 (m, 2H, H-5,6b′), 4.48 (dd, 1H, J=2.0, 12.8 Hz, H-6a), 4.69 (dd, 11H, J=2.5, 12.8 Hz, H-6b), 4.73 (d, 1H, J=8.3 Hz, H-1′), 5.00 (dd, 1H, J=3.3, 10.5 Hz, H-3′), 5.07 (dd, 1H, J=3.8, 10.3 Hz, H-2), 5.25 (dd, 1H, J=8.0, 10.5 Hz, H-2′), 5.49 (d, 1H, J=3.3 Hz, H-4′), 5.63 (t, 11H, J=9.8 Hz, H-3), 6.32 (d, 1H, J=3.5 Hz, H-1); ¹³C NMR (100 MHz, CDCl₃) 630.0, 30.1, 30.2, 30.3 (x2), 30.4, 30.5 (x4), 30.6 (x3), 30.7, 30.8, 31.1 (CH₃×16), 54.4, 54.9, 55.1, 55.2, 55.3, 55.5, 55.6, 56.4 (C-7×8), 62.3 (C-6′), 62.8 (C-6), 68.3 (C-4′), 69.6 (C-2′), 70.2 (C-3), 70.8 (C-5), 70.8 (C-2), 71.0 (C-5′), 72.9 (C-4), 73.0 (C-3′), 89.1 (C-1), 99.6 (C-1′), 169.2 (C═O), 169.3 (C═O), 170.3 (C═O), 170.4 (C═O), 170.5 (C═O), 170.6 (C═O), 170.9 (C═O), 171.0 (C═O);
Preparation of a 3-Arm Star Polymer—Polymer “H”

A Schlenk tube was charged with Cu(I)Br (0.426 g, 2.96×10⁴ mol.), Hostasol Monomer (0.678 g, 1.48×10⁻³ mol.) and 1,3,5-tri-O-isobutyryl bromide benzene (3 arm initiator) (0.564 g, 9.89×10⁴ mol.). The tube was fitted with a rubber septum and pump-filled with nitrogen three times. Deoxygenated and inhibitor-free DMAEMA (46.65 g, 29 mmol), toluene (140 mL), and n-propyl-2-pyridylmethanimine (0.967 g, 5.92×10⁴ mol) were quickly added. The solution was deoxygenated by three freeze-pump-thaw cycles prior to heating to 85° C. under a nitrogen atmosphere with magnetic stirring until 50% monomer conversion had been achieved. The product polymer was passed through a short column of alumina to remove catalyst residues and was isolated by precipitation in to cold petroleum ether and drying in vacuo. Monomer conversion was estimated by ¹H NMR. GPC analysis indicated that the product polymer had an Mₙ value of 6500 g mol⁻¹ and a polydispersity of 1.07.

Preparation of a 5-Arm Star Polymer—Polymer “I”

A Schlenk tube was charged with Cu(I)Br (0.426 g, 2.96×10⁴ mol.), Hostasol Monomer (0.678 g, 1.48×10⁻³ mol.) and 1,2,3,4,6-Penta-O-isobutyryl bromide-α-D-glucose (5 arm initiator)(0.552 g, 5.92×10⁻⁵ mol.). The tube was fitted with a rubber septum and pump-filled with nitrogen three times. Deoxygenated and inhibitor-free DMAEMA (46.65 g, 29 mmol), toluene (140 mL), and n-propyl-2-pyridylmethanimine (0.967 g, 5.92×10⁻⁴ mol) were quickly added. The solution was deoxygenated by three freeze-pump-thaw cycles prior to heating to 85° C. under a nitrogen atmosphere with magnetic stirring until 50% monomer conversion had been achieved. The product polymer was passed through a short column of alumina to remove catalyst residues and was isolated by precipitation in to cold petroleum ether and drying in vacuo. Monomer conversion was estimated by ¹H NMR. GPC analysis indicated that the product polymer had an Mₙ value of 12200 g mol⁻¹ and a polydispersity of 1.09.

Preparation of a 8-Arm Star Polymer—Polymer “J”

The 8-arm star polymer is prepared by following the process steps outlined above for either the 3-arm star polymer or the 5-arm star polymer except that, of course, the 8-arm initiator is used in place of the 3-arm initiator or the 5-arm initiator, respectively.

General Procedure for Quaternisation of pDMAEMA Star Polymers

Polymer (1 g) was dissolved in 20 mL of 1M HCl (aq) with stirring. The orange coloured solution was frozen in liquid nitrogen and freeze-dried to give quaternised pDMAEMA star polymers as brown/orange crystalline solids.

General Procedure for Quaternization of Star Polymer with MeI

Star polymer (10 g) was dissolved in anhydrous THF (500 mL) at room temperature under an atmosphere of nitrogen. Methyl iodide (3.96 ml, 70 mmol) was added and the mixture stirred at room temperature for 12 hours. Precipitation of the polymer starts to occur after ˜20-30 minutes. The reaction mixture was diluted with hexane and the quaternised polymer was filtered and dried in vacuo.

General Procedure for Quaternization of Star Polymer with HCl

Star polymer (11 g) was dissolved in 150 mL of 1M HCl (aq) with stirring. The orange coloured solution was frozen in liquid nitrogen and freeze-dried to give quaternised Hostasol pDMAEMA (12.1 g) as an orange powder.
Synthesis of Hostasol pPEG 475 (DP25)—Polymer “K” 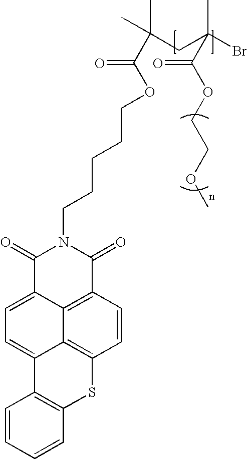
Cu^{(I)}Br (130 mg, 0.909 mmol) and hostasol initiator (490 mg, 0.909 mmol) were weighed out into a schlenk tube and sealed with a subaseal. PEG 475 methylether methacrylate (10 mL, 22.73 mmol, filtered through alumina) and toluene (20 mL) were added via syringe. The mixture was frozen in liquid nitrogen and degassed via four freeze-pump-thaw cycles. The reaction mixture, under an atmosphere of nitrogen, was placed in a oil bath at 90° C. and stirred for 5 minutes. Propyl pyridine-2-yl methyleneamine (propyl ligand) (0.28 mL) was added to initiate polymerisation. The polymerisation mixture was heated to 90° C. under an atmosphere of nitrogen. Reaction aliquots (0.4 mL) were removed at timed intervals. Conversion was followed by ¹H-NMR. After 6.5 hours (100% conversion), the reaction mixture was removed from the oil bath, diluted with toluene (˜20 mL) and filtered through a column of basic alumina to remove the copper catalyst. The column was washed with two column volumes of toluene and toluene/ethyl acetate (1:1, v/v), and the filtrate evaporated under reduced pressure to give an orange coloured oil. The polymer was dissolved in chloroform (˜40 mL) and added dropwise to a beaker of hexane (1.5 L) with vigorous stirring to precipitate the polymer. The solvent was decanted off and the polymer reconstituted in chloroform and evaporated under reduced pressure to give polymer (3.41 g, Mn 14000) as an orange oil.

Synthesis of Hostasol pPEG300 (DP 40)—Polymer “L” 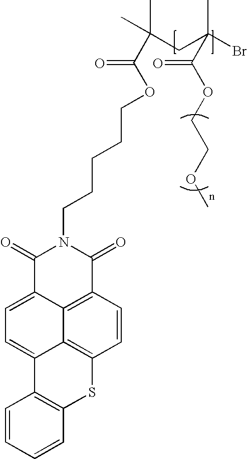
Cu^{(I)}Br (126 mg, 0.875 mmol) and hostasol initiator (470 mg, 0.875 mmol) were weighed out into a schlenk tube and sealed with a subaseal. PEG 300 methylether methacrylate (10 mL, 35 mmol, filtered through alumina) and toluene (20 mL) were added via syringe. The mixture was frozen in liquid nitrogen and degassed via four freeze-pump-thaw cycles. The reaction mixture, under an atmosphere of nitrogen, was placed in a oil bath at 90° C. and stirred for 5 minutes. Propyl pyridine-2-yl methyleneamine (propyl ligand) (0.27 mL, 1.75 mmol) was added to initiate polymerisation. The polymerisation mixture was heated to 90° C. under an atmosphere of nitrogen. Reaction aliquots (0.4 mL) were removed at timed intervals. Conversion was followed by ¹H-NMR.

After 7 hours (100% conversion), the reaction mixture was removed from the oil bath, diluted with toluene (˜20 mL) and filtered through a column of basic alumina to remove the copper catalyst. The column was washed with two column volumes of toluene and toluene/ethyl acetate (1:1, v/v), and the filtrate evaporated under reduced pressure to give an orange coloured oil. The polymer was dissolved in chloroform (˜40 mL) and added dropwise to a beaker of hexane (1.5 L) with vigorous stirring to precipitate the polymer. The solvent was decanted off and the polymer reconstituted in chloroform and evaporated under reduced pressure to give polymer (5.37 g, Mn 14000) as an orange oil.
Synthesis of Hostasol pPEG2000 (DP 15) Polymer “M” 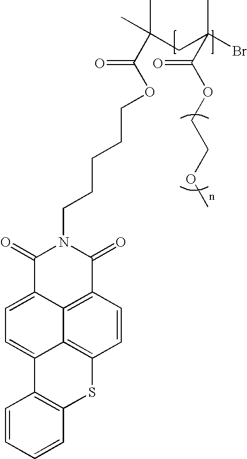
Cu^{(I)}Br (48 mg, 0.33 mmol), hostasol initiator (180 mg, 0.33 mmol) and PEG 2000 methylether (10 g, 5 mmol) were weighed out into a schlenk tube and sealed with a subaseal. Toluene (30 mL) was added via syringe. The mixture was frozen in liquid nitrogen and degassed via four freeze-pump-thaw cycles. The reaction mixture, under an atmosphere of nitrogen, was placed in a oil bath at 70° C. and stirred for 5 minutes. Propyl pyridine-2-yl methyleneamine (propyl ligand) (0.1 mL, 0.66 mmol) was added to initiate polymerisation. The polymerisation mixture was heated to 70° C. under an atmosphere of nitrogen. Reaction aliquots (0.4 mL) were removed at timed intervals. Conversion was followed by ¹H-NMR. After 90 hours, the reaction mixture was removed from the oil bath, diluted with toluene (˜20 mL) and filtered through a column of basic alumina to remove the copper catalyst. The column was washed with two column volumes of toluene and toluene/ethyl acetate (1:1, v/v), and the filtrate evaporated under reduced pressure to give a yellow solid. The polymer was dissolved in chloroform (˜40 mL) and added dropwise to a beaker of hexane (1.5 L) with vigorous stirring to precipitate the polymer. The hexane solution was filtered to give polymer (15.77 g, Mn 14000) as a fine yellow solid.
Preparation of Coumarin-Labelled pDMAEMA—Polymer “D”
Preparation of Hydroxyl Initiator 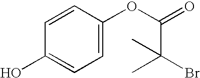
Hydroquinone (110.11 g, 1.0 mol), tetrahydrofuran (800 mL) and triethylamine (15.3 mL, 0.11 mol) were placed in a 1 L round bottom flask equipped with a pressure equilibrating dropping funnel. 2-Bromoisobutyryl bromide (12.4 mL, 0.1 mol) and tetrahydrofuran (87.6 mL) was placed into the dropping funnel. The 2-bromoisobutyryl bromide solution was added dropwise over 2 hours with stirring which formed triethylammonium bromide. Upon complete addition the reaction was stirred for 2 hours. The triethylammonium bromide was removed by filtration and the solvent was removed under reduced pressure. This resulted in a brown white crystalline mixture which was placed into a round bottom flask with chloroform (600 mL) and stirred overnight to separate the unreacted excess hydroquinone. The unreacted hydroquinone was removed from the solution by filtration and the filtrate was concentrated under reduced pressure to give a brown crystalline compound. The resulting brown crystalline solid was purified by column chromatography eluting with 100% dichloromethane followed by 100% methanol to give a pale brown crystalline solid. Yield=18.5 g (71%)
Preparation of Coumarin 343 Initiator 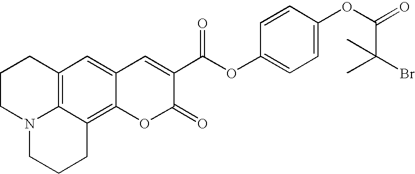
Coumarin 343 (1 g, 3.5 mmol, Exciton Inc.), hydroxyl initiator (0.907 g, 3.5 mmol), N-(3-dimethylaminopropyl)-N′-ethylcarbodiimide hydrochloride (1.34 g, 7.0 mmol) and 4-(dimethylamino)pyridine (0.043 g, 0.35 mmol) were dissolved in anhydrous dichloromethane (25 mL) under nitrogen and stirred at room temperature for 72 hours. The resultant solution was washed with distilled water (2×25 mL), 0.5 M HCl (4×25 mL), saturated NaHCO₃ (4×25 mL), distilled water (2×25 mL) and then dried over MgSO₄. Removal of the solvent in vacuo gave an orange product which was purified by column chromatography on silica gel using 9:1 dichloromethane/ethyl acetate then 9:1 dichloromethane/diethyl ether to give the coumarin 343 initiator in 80% yield. The preparation of the coumarin 343 initiator was adapted from: Sylvain L. Gilat, Alex Adronov, and Jean M. J. Frechet, J. Org. Chem. 1999, 64, 7474-7484.
Preparation of pDMAEMA Polymers Using Coumarin 343 Initiator

Coumarin 343 initiator (0.50 g, 0.95 mmol), Cu(I)Br (0.136 g, 0.95 mmol, 1 eq), DMAEMA (14.2 g, 0.090 mol), toluene (30 mL) and a magnetic follower were placed in an oven dried Schlenk tube. The resulting solution was deoxygenated via three freeze-pump-thaw cycles and then degassed N-propyl-2-pyridylmethanimine (0.28 g, 1.9 mmol) was added. The reaction was placed in a thermostatically controlled oil bath at 90° C. for 4 hours. The polymer solution was diluted with toluene (100 mL) and filtered through a column of basic alumina to remove the catalyst. The column was washed with toluene (2×100 mL) and the combined filtrate was subjected to rotary evaporation to remove the solvent. The polymer was purified twice by precipitation using dichoromethane/petroleum ether to give coumarin 343 initiated pDMAEMA (Mn 12,300, PDi of 1.08).

Quaternisation of pDMAEMA to Give Q10, Q24 and Q32—Polymers “E”, “F” and “G”, Respectively

The polymer was quaternised to obtain 10% (Q10), 24% (Q24) and 32% (Q32) of the repeat units quaternised. In a typical quaternisation, coumarin 343 initiated pDMAEMA (5 g, 0.41 mmol, Mn 12,300, PDi 1.08) was dissolved in tetrahydrofuran (50 ml) and methyl iodide (0.43 g, 3 mmol) was added and the solution left stirring for 24 h at room temperature. The solvent was removed and the polymer dried under vacuum. The degree of quaternisation, as determined by ¹H NMR, was 10% of the available amine groups. This gives a polymer with a number average molecular mass of 13,400.

Assay Methods

Materials

All tissue culture reagents were from Gibco (Biosciences, Ireland). FITC-Dextran (Mw 4400) was from Sigma (Sigma-Aldrich, Ireland). Tissue culture filters and plates were from Corning Costar (Fannin Healthcare, Ireland) and chitosan was obtained from Fluka (Fluka Chemicals, UK). Ethidium homodimer-1 was obtained from Molecular Probes (Molecular Probes Europe BV, The Netherlands). The shaking incubator used in adhesion experiments was a Titramax® 1000 from Heildolph Instruments.

Cell Culture

E12 Cells (Passage 50-57) were a generous gift from Professor Per Artursson, Upsala University, Sweden. HT29 cells were obtained from ATCC (Passage 121-128). Cells were grown and sub-cultured as previously described [Behrens, I., et al., Transport of lipophilic drug molecules in a new mucus-secreting cell culture model based on HT29-MTX cells. Pharm Res. 18: p. 1138-45 (2001)]. Both cell lines were maintained with Dulbecco's Modified Eagles Medium (DMEM) with 10% foetal calf serum (FCS), 1% non-essential amino acids and 1% L-glutamine at 5% CO₂, 95% O₂ at 37° C.

For adhesion, characterisation and transport studies, cells were seeded at a density of 2×10⁴ cells/filter on 12 mm Transwell® polycarbonate membrane inserts (Corning Costar, Cat.# 3401). The cells were fed apically and basolaterally every two days and differentiated to form monolayers over 21 days. Monolayer integrity was examined by measuring Transepithelial Electrical Resistance (TEER) of cell monolayers 21 days post-seeding, and directly before and after adhesion experiments using an EndOhm® electrode system with background correction made for unseeded filters.

Lactate Dehydrogenase Cytotoxicity Assay

Lactate Dehydrogenase (LDH) activity was measured in culture supernatants at 0, 30 and 60 minutes following polymer exposure to cells grown on Transwell® membranes, using a cytotoxicity assay kit (Roche Molecular Biochemicals). LDH concentrations were expressed as percentage LDH release relative to treatment with Triton-X 100 and the percentage cytotoxicity calculated in each case [Korzeniewski, C. and D. M. Callewaert, An enzyme-release assay for natural cytotoxicity. J Immunol Methods. 64: p. 313-20 (1983)].

Polymer Synthesis

Methacrylate-based polymers are quaternised derivatives of 2-(dimethylamino)-methylmethacrylate (DMAEMA) monomer, which were tagged with the fluorescent probes, coumarin or hostasol. Coumarin and hostasol fluoresce in the green spectrum range (400 nm excitation/490 nm emission wavelengths for coumarin fluorescent tags and 465 nm/570 nm for hostasol). They were used to track polymers spectroscopically.

Adhesion Assay: Monolayers

HT29 monolayers (no mucus—used to assess bioadhesion) and E12 monolayers (mucus-covered—used to assess mucoadhesion) were rinsed with HEPES-buffered-DMEM and equilibrated at 37° C. for 60 minutes. In some instances, E12 monolayers were incubated with the mucolytic agent, N-acetylcysteine (10 mM, NAC) for 15 minutes prior to polymer addition, to remove mucus and enable assessment of bioadhesion. To remove the NAC, monolayers were rinsed gently with medium. 0.5 mL of polymer was added to the apical side at two concentrations (0.1 mg/mL or 0.01 mg/mL). Monolayers were incubated at 37° C. in a Titramax® incubator for 60 minutes at 100 rpm. Polymer concentration was measured by sampling from both sides at 0 and 60 minutes. Monolayers were then washed, homogenized, and lysed in 2% SDS 50 mM EDTA at pH 8.0. All samples were assayed on a fluorimeter as described above and expressed as μg polymer per cm² of monolayer/tissue surface area.

Adhesion Assay: Tissue

Rats were starved overnight before euthanasia by cervical dislocation. The intestine was removed after a midline incision, and the jejunum rapidly removed and flushed with oxygenated medium. 6 sacs, each of length 5 cm long, were removed. Sacs were placed in oxygenated medium at 37° C., according to the revised method of Barthe et. al. [An improved everted gut sac as a simple and accurate technique to measure paracellular transport across the small intestine. Eur J Drug Metab Pharmacokinet. 23: p. 313-23 (1998)]. The sacs were tied tightly at one end with silk suture and a small animal vascular catheter (Data sciences international physiocath™ 277-1-002) was tied in to the other end. A 1 mL syringe with a sterile 26 gauge micro lance was attached to the top of the catheter. For assessment of bioadhesion, intestinal sacs were treated with 10 mM NAC and then flushed with 20 mL of medium to remove the NAC and the liquified mucus. For assessment of mucoadhesion, sacs were flushed with medium alone. The sac was then filled with 0.5 mL polymer solution (1 mg/mL) through the catheter. Each sac was placed in a separate sealed 50 ml flask containing 15 mL of oxygenated TC-199 medium on a shaking water bath for 30 minutes at 37° C. Duplicate 50 μL samples of incubation medium were removed after 30 minutes to assess leakage. The sac was then removed from the bath and the contents recovered using a fresh 1 mL syringe. The sac was then washed sequentially four times with a total of 5 mLs of medium via the catheter and the washes collected for assay by fluorimetry as described above.

FD4 Transport Studies

Transport studies were carried out on monolayers and sacs with FITC-Dextran 4400 (FD-4) in order to assess functional integrity. Permeability across monolayers was examined according to the method of Yee [Yee, S., In vitro permeability across Caco-2 cells (colonic) can predict in vivo (small intestinal) absorption in man—fact or myth. Pharm Res. 14: p. 763-6 (1997)]. In brief, 0.5 mL of FD-4 (250 μg/ml) were added to the apical side of the monolayers. FD-4 flux into the basolateral well was measured over 120 minutes. 100 μL: samples were collected from the basolateral side every 15 minutes and subsequently replaced with fresh transport medium. In sac studies, 0.5 mL of FITC-dextran (1 mg/mL) was injected into each sac lumen as described above. 50 μL samples were collected from the bath every 15 minutes and replaced with fresh medium. After 120 minutes the sacs were cut open and the contents sampled. FD-4 was assayed by fluorimetry at excitation wavelength of 490 nm and an emission of wavelength of 525 nm. Apparent permeability (Pₐₚₚ) for monolayers and sacs was calculated from cumulative concentration vs. time profiles using the following equation:
Pₐₚₚ(cm/sec)=(dQ/dt)/(A*Cₒ)

Where dQ/dt (μg/s) represents the cumulative concentration increase in the receiver-side with respect to time, A (cm²) is the surface area of the monolayer or sac and Cₒ (μg) is the initial concentration in the donor compartment.

Confocal Scanning Laser Microscopy (CLSM)

Polycarbonate membranes with attached monolayers were exposed to 0.1% sodium dodecyl sulfate for 2 minutes at room temperature, removed from the insert scaffold and stained with ethidium homodimer-1 (EthD1) (Molecular Probes, catalogue # L-3224). The membranes were placed on glass slides, surrounded by an adhesive compound and covered in 200 μl of DMEM-HEPES transport medium. Glass cover slips were attached, sealed and examined under CLSM (Leica™ TCS SL). The excitation/emission wavelength was 495 nm/635 nm respectively.

Light and Fluorescent Microscopy.

In order to view the mucus gel layer, monolayers grown on transwell membranes were washed in DMEM-HEPES medium, sandwiched between thin strips of chicken liver and snap-frozen in liquid nitrogen. The tissue was embedded in optimal cutting temperature (OCT) compound, cut into 20 μm cross sections with a cryo-microtome and transferred to glass slides for staining. To visualise the mucus gel layer, sections were stained with alcian blue (1%) in distilled water adjusted to pH 2.5 with glacial acetic acid (3%) for 10 minutes at room temperature, washed in a water bath with flowing water. Sections were then counterstained with neutral red (1%) for 5 min at room temperature and washed in a water bath with flowing water. Sections of E12 were examined by light and fluorescent microscopy using a Nikon Eclipse E400 Fluorescent Microscope with Nikon Digital Camera (Model:DXM1200) and captured using Nikon ACT-1 (Version 2.0) imaging software. Slides were visualised in a dual light-fluorescent microscope and a field comparison between light and fluorescent images allowed the visualisation of the fluorescently labelled polymer in the mucus gel layer.

Statistics

Statistical analysis for significance between groups was calculated using students paired or unpaired t-test, as appropriate (GraphPad Instat, Version 3.05). P values of p<0.05 were considered significant.

### EXAMPLE 1

### Co-Localisation of Fluorescent-Tagged Polymer with Mucus on Cultured Cells

Mucoadhesion is the interaction of polymeric substances with glycoproteins in the mucus gel layers overlying the epithelium. Mucoadhesion can occur via 1) covalent, 2) electrostatic, 3) hydrogen bonding and 4) hydrophobic interactions or a combination of these. Potential applications of muco-integrated polymers are for augmenting mucosal barrier function, useful in mucocitis, eye disease and inflammatory bowel diseases. In these conditions, bioadhesion (i.e. adhesion to cells and/or their glycocalyx) and residence times for current therapies are sub-optimal. Mucus binding polymers may also have a role in preventing colonisation and epithelial attachment of mucus-residing bacteria such as Helicobacter pylori and Camphylobacter jejuni.

Fluorescently-labelled polymers useful in the present method were synthesised using living radical polymerisation with good molecular weight control and low polydispersities. Three systems were used as in-vitro models of intestinal epithelium for adhesion experiments: 1) HT29 monolayers (epithelial surfaces), 2) HT29-MTX-E 12 monolayers (mucous-secreting) and 3) rat non-everted intestinal sacs.

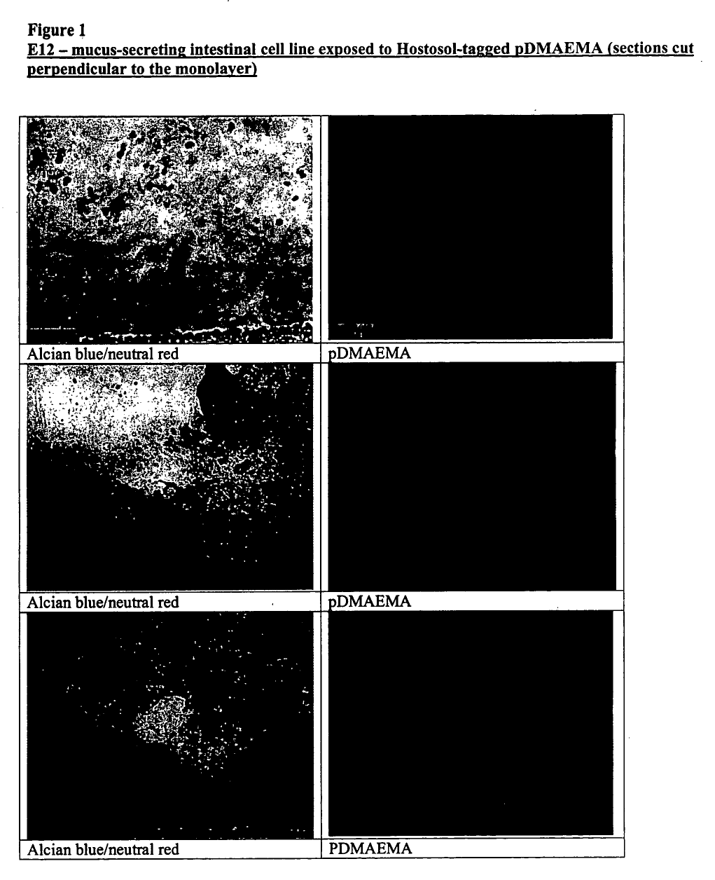

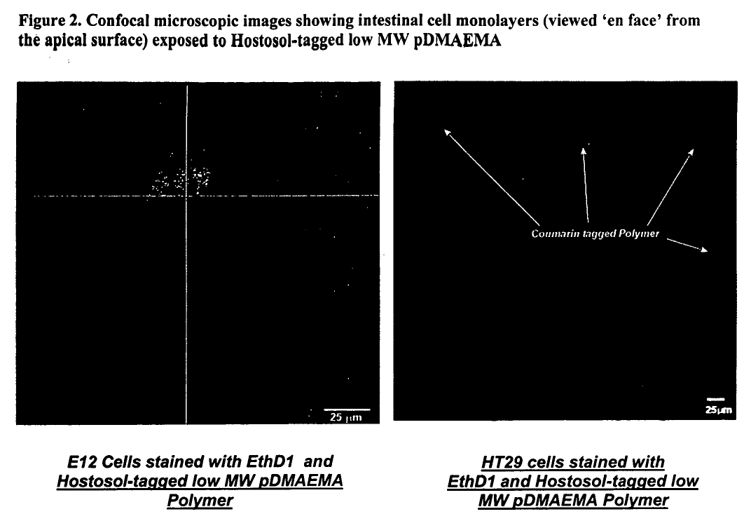

FIGS. 1 and 2 show transmitted light and confocal images showing co-localisation of fluorescent-tagged tagged polymer (green) with mucus on cultured cells (blue—alcian blue stain).

FIG. 1 shows, in adjacent (left vs right) images, snap frozen monolayers of mucus-secreting E12 intestinal cells, grown on transwell membranes, and exposed to pDMAEMA tagged with the Hostosol fluorochrome. These were embedded in liver tissue (to preserve the mucus during processing), snap frozen in liquid nitrogen, and cryosections were cut perpendicular to the membrane. Sections were mounted on slides coated with a tissue adhesive, and stained with Alcian blue (for mucus) and neutral red (for cells). The images on the right are viewed in normal transmitted-light mode, and show the cell monolayer, surmounted by a thick mucus layer of around 100 microns in thickness (stained blue in colour images). The images on the left are the same sections viewed by fluorescent microscopy, and show the (green in colour images) hostosol-tagged polymer integrated into the supramucosal gel overlying the cell monolayer. FIG. 1 shows that the pDMAEMA polymer (Polymer “A”) adheres to, and integrates with, the mucus gel covering the cells.

FIG. 2 shows E12 (mucus covered) vs HT29 (non-mucus covered) cells viewed from the apical surface.

FIG. 2, left-hand image shows E12 cells stained with EthD1 and quaternised Coumarin-tagged low MW pDMAEMA polymer. (Polymer “E”). On colour images, areas of cells (red) can be faintly seen, but are substantially obscured by a granular green ‘wash’ of polymer that is integrated into the mucus layer that surmounts these cells.

FIG. 2, right-hand image, shows HT29 cells stained with EthD1 and quaternised Coumarin-tagged low MW pDMAEMA polymer. (Polymer “E”). On colour images, the predominant colour is red, indicating the cells. A small number of specks (arrows) of adherent polymer a indicate a less even distribution polymer on the cells, which are not mucus-covered.

FIG. 2 shows
The quaternised pDMAEMA polymer forms an adhesive layer on the surface of mucus gels
The adhesion to cells that have no mucus covering appears to be less evenly distributed.

The hydrochloride salt of coumarin-labelled pDMAEMA (Polymer “D”) was found to be as mucoadherent as a gold standard, N-trimethylated chitosan, in the monolayers and the tissue-based system.

### EXAMPLE 2

### pDMAEMA, But not Chitosan, Reduces Flux of the Paracellular Marker FD-4 in Cell Models

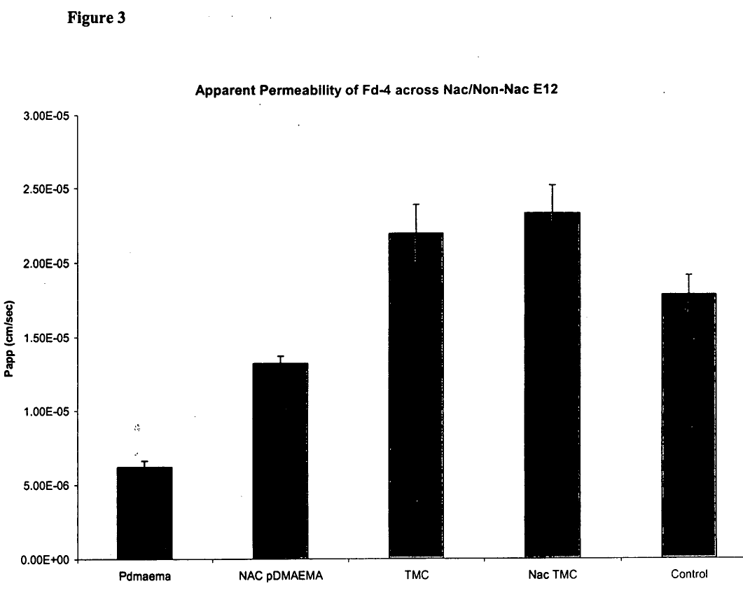

FIG. 3 shows the apparent permeability of FD-4 in the E12 cell model. Notes to FIG. 3:
E12=a mucus-secreting intestinal epithelial cell line.

FD-4=fluorescent tagged dextran 4000, which is a marker used to quantify paracellular flux across epithelial monolayers.
pDMAEMA=polymer (low MW Hostasol, the HCl salt) (Polymer “N”)
TMC=trimethylated chitosan (aqueous soluble at pH 7.4) (Comparative Polymer
Nac=N-acetyl cysteine: used to remove mucus layer from cells to determine its effect on flux data.
Control=untreated E12 cells

One-way Analysis of Variance (ANOVA)

| Test | Significance | P-Value |
|---|---|---|
| PDMAEMA vs TMC | *** | P < 0.001 |
| PDMAEMA vs Nac TMC | *** | P < 0.001 |
| PDMAEMA vs NAC pDMAEMA | Ns | P > 0.05 |
| PDMAEMA vs Control | ** | P < 0.01 |
| TMC vs Nac TMC | Ns | P > 0.05 |
| TMC vs NAC pDMAEMA | Ns | P > 0.05 |
| TMC vs Control | Ns | P > 0.05 |
| Nac TMC vs NAC pDMAEMA | * | P < 0.05 |
| Nac TMC vs Control | Ns | P > 0.05 |
| NAC pDMAEMA vs Control | Ns | P > 0.05 |

Conclusions:
Removal of the mucus gel layer using NAC causes an increase in permeability to FD4 in all cases
TMC enhances the permeability of the cell monolayer above controls in all cases
pDMAEMA polymer (Polymer “N”) substantially reduces permeability over controls. This is especially the case where the mucus layer is intact (indicating a specific interaction between the polymer and the mucus gel)
pDMAEMA (Polymer “N”) has different pharmacological properties to chitosan.

### EXAMPLE 3

### Rank Order of Polymers in Cells and Tissue: Comparison of Polymers D, E, F, G, H and I with TMC as an Industry Standard

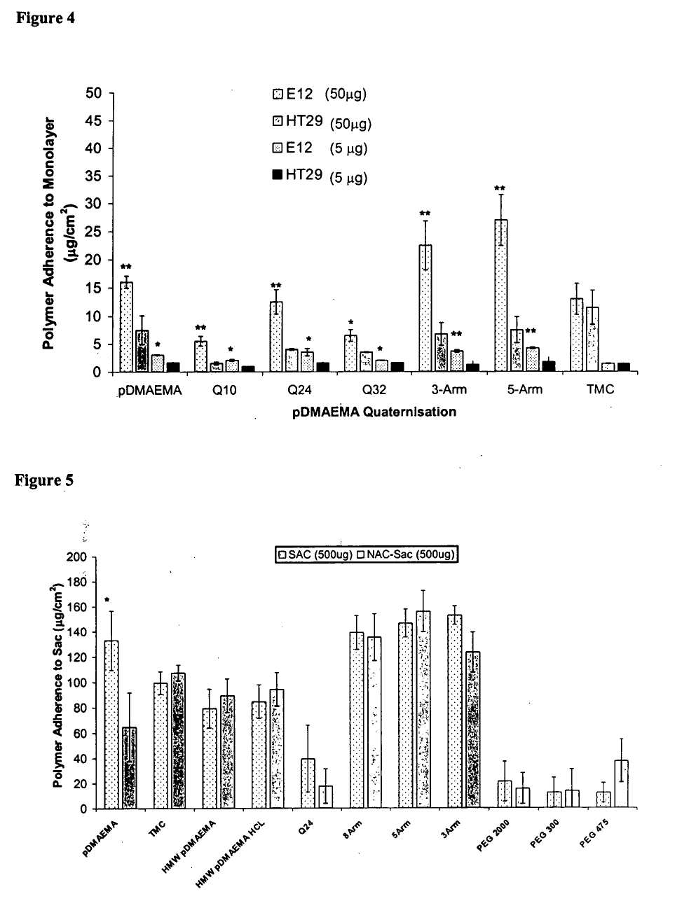

FIG. 4 shows polymer adherence to an E12 or HT29 monolayer and its relationship to pDMAEMA quaternisation (comparison of Polymer “D” (unquaternised) with Polymers “E”, “F” and “G”. Adhesion of polymers to E12 (mucus secreting) vs HT29 (non-mucus secreting) cells are shown at 2 different dosage levels (assay volume 500111) (FIG. 4). Notes to FIG. 4:

*=significantly greater adhesion to mucus covered cells vs non-mucus covered cells

**=highly significantly greater adhesion to mucus covered cells vs non-mucus covered cells

Conclusions:

Enhancement of the positive charge of the low molecular weight pDMAEMA polymer by quaternerisation does not substantially affect adhesion
Adhesion of pDMAEMA 3-Arm star (Polymer “H”), and 5 arm star (Polymer “I”) to mucus-secreting (E12) cells is greater than that for TMC at 2 different dose levels.
All of Polymers D, E, F, G, H and “I” (especially the multi-arm low MW forms i.e. Polymers “H” and “I”) adhere preferentially to mucus, whereas TMC adheres equally to cells and mucus.

The use of bioadhesives (to cells) and mucoadhesives (to mucus) offers advantages in improving interaction with mucosal surfaces. While there is no established means of measuring intestinal mucoadhesion, HT29-MTX-E 12 (MTX-E 12), a recent mucous-secreting human intestinal carcinoma cell line [Behrens, I., et al., Transport of lipophilic drug molecules in a new mucus-secreting cell culture model based on HT29-MTX cells. Pharm Res, 2001. 18(8): p. 1138-45], offers a reductionist model for mucoadhesion in the gut.

Filter grown-MTX-E12 monolayers and non-everted rat intestinal sacs were apically exposed to fluorescently labelled polymer at varying concentrations for 60 minutes under physiological conditions. Quaternised derivatives of pDMAEMA, a tertiary amino methacrylate, and a known bioadhesive, N-trimethylated chitosan (TMC) [Thanou, M., J. C. Verhoef, and H. E. Junginger, Chitosan and its derivatives as intestinal absorption enhancers. Adv Drug Deliv Rev, 2001. 50 Suppl 1: p. S91-101] were tested in this manner and ranked in order of adhesiveness. Both systems were treated with N-acetyl-cysteine (N-ac), a known mucolytic agent [Behrens, I., et al., Comparative uptake studies of bioadhesive and non-bioadhesive nanoparticles in human intestinal cell lines and rats: the effect of mucus on particle adsorption and transport. Pharm Res, 2002. 19(8): p. 1185-93], as a non-mucus-covered control for the assessment of adhesion. Non-mucous-secreting HT29 monolayers were also used as an independent non-mucus-covered control. pDMAEMA and its quaternised derivatives showed significantly more adhesion in the presence of a mucous gel layer in both systems. TMC showed equal levels of bioadhesion and mucoadhesion. Ranking was quantified in the MTX-E12 model and the degree of pDMAEMA quaternisation showed no discernable relevance to the level of polymer adhesion. The non-everted sac model showed comparable results in polymer adhesion with and without mucous gel layer. 24% quaternised pDMAEMA showed the highest level of adhesion overall, followed by un-quaternised pDMAEMA, TMC, 10% quaternerised pDMAEMA and 32% quaternerised pDMAEMA. This indicates that ionic charge may not the predominant factor in the adhesion of pDMAEMA and that the mechanism of mucoadhesion is comparable and quantifiable in two unique systems.

FIG. 5 shows adhesion of various forms of pDMAEMA and TMC to the lumenal surface of isolated rat intestinal sacs (assay volume 500 μl, polymer conc. 1 mg/ml). Notes (FIG. 5):
Sac=untreated mucosal surface

NAC-Sac=sac in which mucus has been removed using treatment by N-acetyl cysteine

| Name Used | Tag | Polymer |
|---|---|---|
| PDMAEMA | Coumarin | Unquaternized poly(2-(dimethylamino- |
| “D” | | ethyl) methacrylate) (pDMAEMA) |
| Q10 “E” | Coumarin | 10% quaternised pDMAEMA |
| Q24 “F” | Coumarin | 24% quaternised pDMAEMA |
| Q32 “G” | Coumarin | 32% quaternised pDMAEMA |
| 10% Dex | Hostosol | PDMAEMA quaternised to 10% with |
| | | Dexamethasone Bromoacetate |
| 20% Dex | Hostosol | pDMAEMA quaternised to 20% with |
| | | Dexamethasone Bromoacetate |
| TMC “Z” | Coumarin | N-Trimethylated-Chitosan |
| HMW | Hostosol | High Molecular Weight pDMAEMA |
| pDMAEMA | | |
| “B” | | |
| HMW | Hostosol | High Molecular Weight pDMAEMA |
| pDMAEMA | | Hydrochloride Salt |
| HCL “C” | | |
| 8Arm “J” | Hostosol | 8 Arm pDMAEMA Polymer |
| 5Arm “I” | Hostosol | 5 Arm pDMAEMA Polymer |
| 3Arm “H” | Hostosol | 3 Arm pDMAEMA Polymer |
| PEG 2000 | Hostosol | Poly Ethylene Glycol 2000, |
| “M” | | coupled to pDMAEMA |
| PEG 300 “L” | Hostosol | Poly Ethylene Glycol 300, |
| | | coupled to pDMAEMA |
| PEG 475 “K” | Hostosol | Poly Ethylene Glycol 475, |
| | | coupled to pDMAEMA |

Conclusions
pDMAEMA (Polymer “D”) is more mucoadhesive (to mucus), but less bioadhesive (to cells) than TMC
high MW pDMAEMA (Polymers “B” and “C”) display approximately equal mucoadhesion and bioadhesion, which is only slightly less than TMC
Star-configured forms of pDMAEMA (Polymers “H”, “I” and “J”) are substantially more mucoadhesive than TMC
PEGylation (Polymers “K”, “L” and “M”) substantially diminishes the mucoadhesion of pDMAEMA.

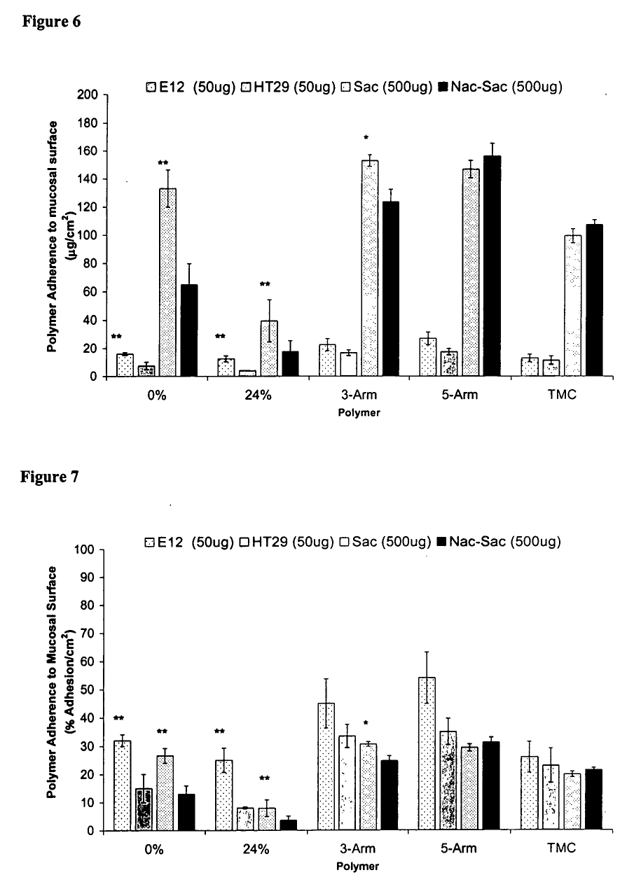

FIG. 6 shows adhesion of pDMAEMA to the lumenal surface of isolated rat intestinal sacs vs mucus covered (E12) and non-mucus-covered HT29 cells (Mass adhesion). Notes to FIG. 6:
Sac=untreated mucosal surface
NAC-Sac=sac in which mucus has been removed using treatment by N-acetyl cysteine
The chart compares mass adherence at different dosage levels in an assay volume of 500 μl
0%=pDMAEMA (unquaternerised—Polymer “D”)
24%=Polymer “F”
3-arm and 5-arm=Polymers “H” and “I” respectively
*=significantly greater adhesion to mucus covered cells vs non-mucus covered cells
**=highly significantly greater adhesion to mucus covered cells vs non-mucus covered cells
   Conclusions:
Enhancement of the positive charge of the pDMAEMA polymer by quaternisation does not substantially affect adhesion in a cell culture system, but 24% quaternisation reduces both muco- and bio-adhesion to isolated intestinal tissue by more than 50%.
The capacity of pDMAEMA polymers to adhere to whole mucus-covered intestinal tissue is substantial, and is greater than TMC for all but the 24% quaternised pDMAEMA.
Increasing the concentration of the polymer also increases the adhesion of the polymer, but this effect is not in direct proportion to the difference in polymer concentration.

FIG. 7 shows adhesion of pDMAEMA to the lumenal surface of isolated rat intestinal sacs vs mucus covered (E12) and non-mucus-covered HT29 cells (% adhesion). Notes to FIG. 7:
The data are the same as shown in FIG. 6 but, instead of total mass of adherent polymer, it is expressed as a percentage of the total polymer added
Sac=untreated mucosal surface
NAC-Sac=sac in which mucus has been removed using treatment by N-acetyl cysteine
The chart compares mass adherence at different dosage levels in an assay volume of 500 μl
0%=pDMAEMA (unquaternerised)
24%=Polymer “F”
3-arm and 5-arm=Polymers “H” and “I” respectively
*=significantly greater adhesion to mucus covered cells vs non-mucus covered cells
**=highly significantly greater adhesion to mucus covered cells vs non-mucus covered cells
   Conclusions:
The adhesion of polymer expressed as a % of that added to the experiment is similar in both cell culture and whole tissue assays
When an equivalent mass of polymer is loaded into experiments using either intestinal cells or whole intestinal tissue, mucoadhesion expressed as a percentage of the total adherent polymer is greater for all of the tested forms of pDMAEMA than for TMC, the only exception being the 24% quaternised pDMAEMA
When an equivalent mass of polymer is loaded, using either intestinal cells or whole intestinal tissue, bioadhesion expressed as a percentage of the total adherent polymer is greater for the 3- and 5-star polymer forms of pDMAEMA than for TMC

Comparison of adhesion of pDMAEMA and trimethylated chitosan to a mucus-secreting cell line (E12) and rat intestinal mucosal tissue (ex vivo)

| | | | 24% Quaternised | | | |
|---|---|---|---|---|---|---|
| | PDMAEMA | | pDMAEMA | | | |
| | | Mass | | Mass | Tri-methylated Chitosan | |
| | Adhesion/ | adhesion | Adhesion/ | adhesion | Adhesion/ | Mass adhesion |
| | cm² | [symbol] | cm² | [symbol] | cm² | [symbol] |
| E12 | 32 ± 6.0% | 16 ± 3.0 | 25 ± 4.0% | 12.5 ± 2 | 26 ± 5.5% | 13 ± 3 |
| monolayers | | | | | | |
| (n = 8) | | | | | | |
| N-Ac Treated E12 | 17 ± 7.0% | 8.5 ± 3.5 | 12 ± 0.7% | 6 ± 0.4 | 22 ± 5.0% | 11 ± 2.5 |
| (n = 8) | | | | | | |
| HT29 (n = 8) | 15 ± 5.0% | 7.5 ± 2.5 | 8 ± 0.3% | 4 ± 0.2 | 23 ± 6.0% | 11.5 ± 3 |
| | N = 4 | | N = 5 | | N = 8 | |
| Rat mucosa | 27 ± 5.0% | 133 ± 5 | 8 ± 5.0% | 39 ± 5 | 20 ± 2.0% | 99 ± 2 |
| N-Ac Treated | 13 ± 5.0% | 65 ± 5 | 4 ± 3.0% | 18 ± 3 | 21 ± 1.0% | 107 ± 1 |
| Non-Everted Sac | | | | | | |

A Comparison of Polymer Adhesion/Cm after 60 minute incubation.
Notes:
E12 and HT29 polymer dosage was 50 μg (in 500 μl),
Non-everted sac polymer dosage was 500 μg (in 500 μl).

N—Ac=N-acetyl cysteine: used to remove mucus layer from cells to determine its effect on adhesion
PDMAEMA=Polymer “D”
24% quaternised pDMAEMA=Polymer “F”
   Conclusions:
Low MW PDMAEMA (Polymer “D”) that is non-quaternerised is more adhesive to mucus covered cell monolayers and mucosal tissue than TMC
TMC is more adhesive than low MW PDMAEMA (Polymer “D”) when the mucus covering of the cells or mucosal tissue is removed by N—Ac
Low MW PDMAEMA (Polymer “D”) has a proportionately greater adhesion to mucus, rather than cells, when compared to TMC.

### EXAMPLE 4

### pDMAEMA Impedes Cell Damage Caused by Cholera Toxin B

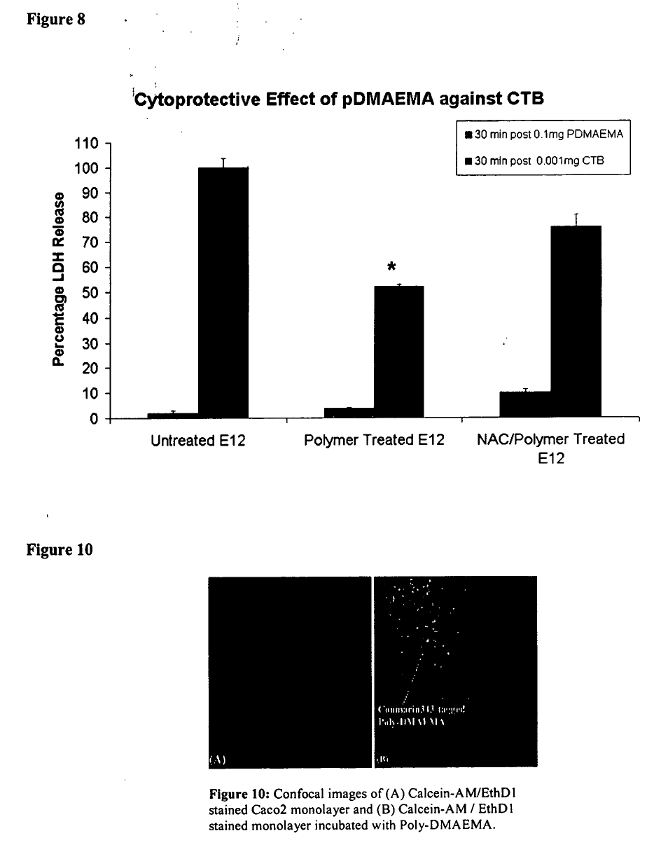

FIG. 8 shows the cytoprotective effect of pDMAEMA (Polymer “D”) against cholera toxin B (CTB). Notes to FIG. 8:
E12=mucus-covered cell line,
N—Ac=N-acetyl cysteine: used to remove mucus layer from cells to determine its effect on adhesion
Toxicity is determined by the % release of a cytosolic enzyme (Lactate dehydrogenase=LDH) from the cells.
   Conclusions:
pDMAEMA (Polymer “D”) substantially enhances (˜50%) the barrier to toxin damage in both mucus-covered and mucus-denuded cells.
The effect of the polymer is substantially greater when the mucus layer of the cells is intact.
pDMAEMA displays very low levels of toxicity to the cells when they are treated with NAC to remove mucus. This effect is negligible in mucus-covered cells.

### EXAMPLE 5

### The Role of the Mucous Gel Layer in Bio-Adhesion of Polymers to Epithelial Cell Monolayers

The small intestine is selectively permeable, acting as a barrier to permeation of toxic compounds and macromolecules. During intestinal injury and inflammation, mucosal absorption of normally-excluded substances can increase dramatically. The identification of the bioadhesive polymers used herein have implications in the treatment of a variety of intestinal disorders.

The HT29-MTX-E 12 (E 12) cell line is a sub-clone of methotrexate-differentiated HT29 human colorectal adenocarcinoma cells. They form confluent monolayers of differentiated intestinal epithelial cells with tight junctions and microvilli, and have been shown to possess an overlying mucous gel layer of 150 μm. To investigate the effect of the mucous gel on bio-adhesion, E12 and HT29 (the non-mucous secreting parent line) cells were seeded on polycarbonate Transwell™ filters (Costar, catalogue #3401) and maintained as previously described (Behrens, I., P. Stenberg, Artursson, P., Kissel, T. Transport of lipophilic drug molecules in a new mucus-secreting cell culture model based on HT29-MTX cells.” Pharm. Res., 2001; 18: 1138-45). The apical sides of the monolayers were then exposed to Poly-DMAEMA (Polymer “D”) at concentrations of 0.01 mg/ml, 0.1 mg/ml and 1 mg/ml (N=6 for each) for 60 minutes on a shaking incubator at 37° C. (Titramax®). Following washing of the monolayer, homogenates were fluorometrically examined for polymer attachment. These data are shown in Table 1:

TABLE 1
Attachment of Poly-DMAEMA

| Polymer | | | |
|---|---|---|---|
| Concen- | | | |
| tration | HT29 | E12 | P value |
| 0.01 mg/ml | 4 ± 0.1 [symbol] (40 ± 11%) | 7 ± 0.11 (70 ± 11%) | <0.02 |
| 0.1 mg/ml | 15 ± 51 [symbol] (15 ± 5%) | 33 ± 61 (33 ± 6%) | <0.0015 |
| 1.0 mg/ml | 6 ± 2% | 14 ± 5% | <0.0015 |

Bioadhesive polymer attachment was approximately 50% higher in E112 than HT29. After polymer exposure for 60 minutes, monolayers were assayed for LDH release (Roche Diagnostics LDH kit). The percentage cytotoxicity for HT29 was 4% at 0.01 mg/ml, 5% at 0.1 mg/ml and 9% at 1 mg/ml. For E12, the percentage cytotoxicity was 1% at 0.01 mg/ml, 3% at 0.1 mg/ml and 3% at 1 mg/ml (N=6, P<0.05 at each concentration). The higher cytotoxicity levels at all polymer concentrations for the HT29 over E12 suggests that mucous may offer protection against cytotoxic effects on epithelial cell monolayers. The in vitro reductionist model of intestinal epithelium described here displays physiological parameters relating to mucous coverage found in vivo.

Cell viability was assessed by measuring LDH leakage. Measurement of LDH leakage showed that E 12 monolayers had up to a 3% loss of cell viability after exposure to polymer, while HT29 monolayers had a cell viability loss twice as high (P<0.02). After polymer exposure for 60 minutes, transport studies were performed on cell monolayers. The paracellular marker, FITC-Dextran (FD-4) was incubated apically, at a concentration of 250 μg/ml and the flux across the monolayer measured. Permeability coefficients (Pₐₚₚ) of FD-4 were calculated. Pₐₚₚ for unexposed E112 and HT29 monolayers were compared to monolayers treated with pDMAEMA. A two-fold decrease in Pₐₚₚ of FD4 for pDMAEMA-treated E12 against untreated E12 monolayers (P<0.02) was shown. No change was seen for HT29 monolayers.

The data suggest that mucous performs a cytoprotective function in the E12 model. The decrease in FD-4 flux in the presence of polymer, may be due to improvement in the physical barrier offered by muco-integrated pDMAEMA. Due to their ability to target and localise to mucous-covered epithelia, muco-integrating biopolymers may have potential as a therapeutic strategy against disorders such as inflammatory bowel disease (IBD).

### EXAMPLE 6

### Interaction of a Hydrophilic Polymer with Mucous-Secreting Intestinal Epithelial Cell Monolayers

Bioadhesion assays were carried out on two types of human cultured intestinal epithelial cell monolayers, mucous-secreting HT29-MTX-E12 and non-mucous-secreting Caco-2. The presence of a mucosal gel layer resulted in up to 5 times more adhesion of a polymer, which was accompanied by a 6-8-fold reduction in the loss of transepithelial resistance.

Caco-2 monolayers consist of differentiated polarized enterocytes connected by tight junctions. Despite their use in in vitro drug transport studies, Caco-2 monolayers lack mucous-secreting goblet cells (Artursson et al., Advanced Drug Delivery Reviews, 22:1655-1658 (1997). Another human intestinal cell line, HT29, expresses mature goblet cells in the presence of methotrexate (MTX) selection pressure (Le Suffleur et al., Cancer Res., 50:6334-6343 (1990). These HT29-MTX subclones have been isolated and characterized (Behrens et al., Pharm. Res., 18:1138-1145 (2001)). Furthermore, it was shown that a mucous gel layer covers the monolayer and is a significant barrier to the permeation of selected drugs and to the uptake of hydrophobic nanoparticles (Behrens et al., Pharm. Res., 18:1138-1145 (2001), Behrens et al., Pharm. Res., 19:1185-1193 (2002)). HT29-MTX-E12 have been shown to have a mucous gel layer thickness of 142±51 μm (Behrens et al., Pharm. Res., 18:1138-1145 (2001)), which corresponds favourably with in vivo measurements taken from human small intestine. This makes HT29-MTX-E12 a useful in vitro reductionist model for intestinal mucoadhesion and drug permeation.

This example investigates the role of the mucous gel layer in the adhesion of a fluorescently-labelled hydrophilic polymer and, more specifically, adherence to Caco-2 and the HT29 sub-clone, while assessing consequences of the interaction visually and electrically.

Experimental Methods

Cell Culture

Caco-2 and HT29-MTX-E12 cells were cultured according to previous methods using supplemented DMEM medium (Artursson et al., Advanced Drug Delivery Reviews, 22:1655-1658 (1997), Behrens et al., Pharm. Res., 18:1138-1145 (2001)). At confluence, cells were seeded at a density of 6×10 5 cells/well on polycarbonate Transwell™ filter inserts (Costar catalogue # 3401). The cells were fed every second day and differentiated over 21 days.

Transepithelial Electrical Resistance (TEER)

Monolayer integrity was examined by measuring TEER of cell monolayers 21 days post-seeding, and directly before and after adhesion experiments using an EndOhm electrode system with background correction made for unseeded filters. TEER decrease may be caused by several factors including opening of tight junctions or cytotoxicity. Prior to the adhesion experiment, the Caco-2 TEER values were 1033±60 Ω.cm², and HT29-MTX-E12 were 566±48 Ω.cm². After 60 minutes, untreated monolayers of both type showed an average of 3% decrease in TEER. After 60 minutes, exposure to 0.65 mg/ml poly-DMAEMA, the TEER decrease was 37±2% in Caco-2 and 16±5% in HT29-MTX-E12 (P<0.02). 0.13 mg/ml of polymer caused a decrease of 14±3% in Caco2 and 4±2% in HT29MTX-E12 (P<0.02). The absolute TEER values of all monolayers before and after treatment remained within acceptable parameters, indicating a viable monolayer (Behrens et al., Pharm. Res., 18:1138-1145 (2001)).

Adhesion of Poly-DMAEMA to Monolayers

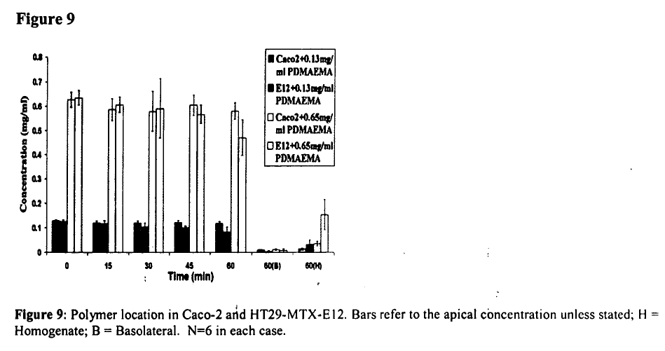

The hydrophilic polymer was a 2-(dimethylamino)-ethylmethacrylate (DMAEMA) monomer construct tagged with coumarin 343 (MW˜12,300—Polymer “D”). Monolayers were rinsed with HEPES-buffered-HBSS transport medium and allowed to equilibrate at 37° C. for 30 minutes. 0.5 ml of polymer was added to the apical side at two concentrations (0.13 mg/ml or 0.65 mg/ml). Monolayers were incubated at 37° C. in a Titramax incubator for 60 minutes at 100 rpm. Polymer concentration was measured by sampling from the apical side at 0,15, 30, 45 and 60 minutes, and from the basolateral side at 60 minutes. At this point the monolayers were washed, homogenized, and lysed in 2% SDS/50 mM EDTA at pH 8 (Behrens et al., Pharm. Res., 19:1185-1193 (2002)). All samples were assayed on a fluorimeter at excitation/emission wavelengths of 400 nm/490 nm. FIG. 9 shows polymer location in Caco-2 and HT29-MTX-E12. HT29-MTX-E12 took up the polymer at both concentrations over 60 minutes, reflecting gradual loss from the donor/apical-compartment. At 60 minutes, the HT29-MTX-E12 homogenate contained an average of 24±4% of the initial concentration (FIG. 9). Caco2 cell homogenate uptake was only 5±2% at 0.65 mg/ml (P<0.02) and 11±2% uptake at 0.13 mg/ml (P<0.02). These concentration-dependent uptake values reflected a loss from the donor compartment, with transepithelial transport being minimal across both systems. A mass balance was present for Polymer “D” in both systems.

Confocal Scanning Laser Microscopy (CLSM)

Polycarbonate membranes with attached Caco-2 monolayers were removed from the inserts and treated with calcein-AM and ethidium homodimer-1 (EthD1) (Molecular Probes, catalogue # L-3224). The membranes were placed on glass slides, surrounded by an adhesive compound and covered in 200%1 of HBSS transport medium. Glass coverslips were attached, sealed and examined under CLSM (Leica™ TCS SL). Excitation/emission wavelengths were as recommended by the manufacturer. FIG. 10 shows confocal images of (A) Calcein-AM/EthD1 stained Caco2 monolayer and (B) Calcein-AM/EthD1 stained monolayer incubated with poly-DMAEMA—Polymer “D”. Adherent polmyer is indicated by a lighter granular material, which is bright green in colour images, and surmounts the underlying red-stained cells. Please note that the cell monolayer is viewed en-face from the apical surface. Stained Caco-2 cells were examined by CLSM after exposure to polymer. Polymer appeared to bind to Caco-2 in a punctate irregular manner (FIG. 10). Even though the polymer showed partial association with the Caco-2 monolayer, only a low concentration was detected in homogenate after washing.

A 5-fold increase in uptake of a hydrophilic polymer was detected in mucous-secreting HT29-MTX-E12 over non-mucous secreting Caco-2. Poly-DMAEMA Polymer “D” caused TEER reduction in both cell lines. In HT29-MTX-E12 cells, the effect of the polymer on TEER reduction was less than in Caco-2. The data suggest that the mucosal gel layer may provide protection against potential cytotoxic effects of poly-DMAEMA.

### EXAMPLE 7

### The Effect of Quaternisation on the Adhesion of a Hydrophilic Polymer

The effect of quaternisation on the adhesiveness of pDMAEMA, a hydrophilic polymer, was studied in various human intestinal epithelial cell culture and excised tissue models. Results showed that quaternisation did not influence the level of pDMAEMA adhesion, which was equal to the adhesion of tri-methylated chitosan (TMC), a known bioadhesive, in the presence of a mucous gel layer.

This example investigates the effects of quaternisation on the muco and bio-adhesive properties of pDMAEMA, a fluorescently-labelled hydrophilic polymer (Polymer “D”) and, more specifically, adherence to the HT-29 parent line, the E12 sub-clone and mucus stripped and non-mucus stripped excised tissue while comparing performance against a tri-methylated chitosan (TMC) polymer.

Cell Culture

HT29 and HT29-MTX-E12 cells were cultured according to previous methods using supplemented medium. At confluence, cells were seeded at a density of 6×10 5 cells/well on polycarbonate Transwell™ filter inserts (Costar catalogue # 3401). The cells were fed every second day and differentiated over 21 days.

Adhesion of pDMAEMA and TMC to Monolayers

The hydrophilic polymers were quaternarised derivatives of a 2-(dimethylamino)-ethylmethacrylate (DMAEMA) monomer construct tagged with coumarin 343 (MW˜12,300). TMC was synthesized as taught above under “Methods”. Monolayers were rinsed with HEPES-buffered-DMEM transport medium and allowed to equilibrate at 37° C. for 60 minutes. 0.5 ml of polymer was added to the apical side at two concentrations (0.1 mg/ml or 0.01 mg/ml). Monolayers were incubated at 37° C. in a Titramax® incubator for 60 minutes at 100 rpm. Polymer concentration was measured by sampling from the apical side at 60 minutes, and from the basolateral side at 60 minutes. At this point the monolayers were washed, homogenized, and lysed in 2% SDS/50 mM EDTA at pH 8. All samples were assayed on a fluorimeter at excitation/emission wavelengths of 400 nm/490 nm.

Adhesion of pDMAEMA and TMC to Tissue

Rats were starved overnight before euthanasia by cervical dislocation. The intestinal mass was removed after a midline incision, and the jejunum rapidly removed and flushed with oxygenated medium, observing minimal handling. Approx 6 sacs, each 5 cm long, were prepared by dividing the intestine at 5 cm intervals and maintained in oxygenated medium at 37° C. The sacs were tied tightly at one end with silk suture and a small animal vascular catheter (data sciences international physiocath™ 277-1-002) was tied in to the other end. A 1 ml syringe with a sterile 26 gauge micro lance was attached to the top of the catheter. For non-mucous gel layer controls, sacs were pre-treated with N-acetyl-cysteine, a known mucolytic, for 15 minutes at 37° C. and then flushed. The sac was then filled with polymer through the catheter, ensuring that the entire loading volume of 0.5 ml entered the sac and that no air pockets were present. Each final sac was placed in a separate 50 ml flask, containing 15 ml of oxygenated TC-199 medium at 37° C., within 18 mins of death. A rubber bung was used to seal the flask and the sac was incubated on a shaking water bath over 30 minutes. Duplicate 50 ul samples of the incubation medium were removed after 30 minutes to assay for leakage of the sac. The sac was then removed from the bath and the contents recovered using a fresh 1 ml syringe. The sac was then washed sequentially four times with a total of 5 mls of medium via the catheter. The wash was collected for assay.

Results and Discussion

Adhesion of pDMAEMA and TMC to Monolayers

Although polymer uptake occurred in all systems, reflected by a gradual decrease of polymer concentration in the apical/donor compartment, there was no obvious proportional relationship between the degree of pDMAEMA quaternisation and the level of adhesion observed in human intestinal epithelial monolayers (HT29 and E12).

TABLE 1
Mass adhesion of polymer in [symbol] of monolayer
(total mass added in brackets) (n = 12)

| % Quater- | | | | |
|---|---|---|---|---|
| nisation | [symbol] | [symbol] | [symbol] | [symbol] |
| 0 | 16.0 ± 3.05 | 7.50 ± 2.55 | 3.00 ± 0.05 | 1.65 ± 0.05 |
| 10 | 5.50 ± 0.90 | 1.50 ± 0.25 | 2.05 ± 0.15 | 0.95 ± 0.05 |
| 24 | 12.5 ± 2.15 | 4.00 ± 0.15 | 3.50 ± 0.65 | 1.55 ± 0.15 |
| 32 | 6.50 ± 1.05 | 3.50 ± 0.05 | 2.00 ± 0.05 | 1.60 ± 0.05 |
| TMC | 13.0 ± 2.75 | 11.5 ± 3.05 | 1.40 ± 0.05 | 1.40 ± 0.05 |

All pDMAEMA derivatives had a significantly stronger (P<0.01 in all cases) association to the cell monolayer in the presence of mucus. This was less evident with TMC, which showed comparable levels of monolayer association in the presence and absence of mucus (Table 1). The un-quaternised and 24% quaternised were found to be as mucoadhesive as TMC but not as bioadhesive.

The quaternisation of pDMAEMA does not influence the level of adhesion in cell or tissue culture intestinal models, with the unquaternised and 24% quaternised showing the highest level of adhesion. pDMAEMA adhesion may be based on non-electrostatic interactions with the mucosal layer. Excised tissue showed roughly twice the level of adhesion of cell culture models in both the presence and absence of a mucus gel layer. pDMAEMA shows comparable mucoadhesive properties to TMC and may have potential in mucosal localisation of therapeutics.

1. A method of augmenting an epithelial mucosal barrier, the method comprising contacting the epithelial mucosal barrier with a topical composition comprising, dispersed in a topically acceptable vehicle, at least one polymer either
a containing a repeat unit of structure 1 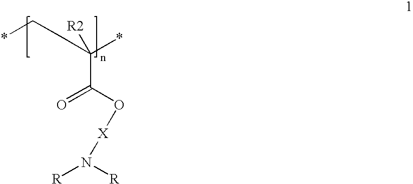
in which each R is independently H, a lower C1-12 straight or branched alkyl, a lower C2-12 alkenyl or a lower C2-12 alkynyl or a substituted or unsubstituted aryl or both R together form a cyclic or a heterocyclic ring; R2 is H or a lower C1-12 straight or branched alkyl; X is a bivalent radical selected from a C1-5 alkylene, C2-5 alkenylene or C2-5 alkynylene radical which may be interrupted by a heterocyclic atom; and n is between 2 and 2000; or
b. containing a repeat unit having the structure 2 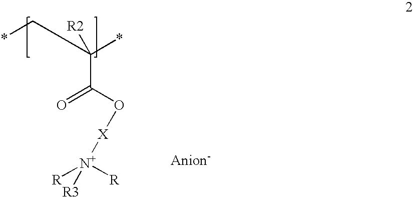
in which R, X, R2 and n are as defined above; and R3 is H, lower C1-12 alkyl, lower C2-12 alkenyl or lower C2-12 alkynyl or a substituted or unsubstituted aryl; and anion is a monovalent anion;
or a mixture of repeat units having structures 1 and 2.

2. A method according to claim 1, in which the at least one polymer is dissolved in the topically acceptable vehicle.

3. A method according to claim 1, in which the at least one polymer has a polydispersity index between about 1.00 and about 5.00.

4. A method according to claim 1, in which the weight average molecular weight of the at least one polymer is less than about 500,000.

5. A method according to claim 3, in which the weight average molecular weight of the at least one polymer is less than about 500,000.

6. A method according to claim 1, in which each R is independently hydrogen or a C1-2 lower alkyl; R2 is hydrogen or a C1-2 lower alkyl; and X is a C1-2 lower alkylene radical.

7. A method according to claim 1, in which each R is independently methyl; R2 is methyl; and X is an ethylene radical.

8. A topical composition for topical application to a mucosal surface, the topical composition comprising, dispersed in a topically acceptable vehicle, at least one polymer either
a. containing a repeat unit of structure 1 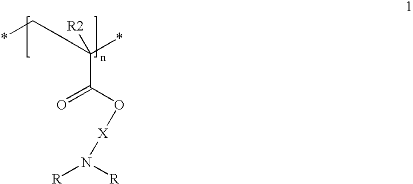
in which each R is independently H, a lower C1-12 straight or branched alkyl, a lower C2-12 alkenyl or a lower C2-12 alkynyl or a substituted or unsubstituted aryl or both R together from a cyclic or a heterocyclic ring; R2 is H or a lower C1-12 straight or branched alkyl; X is a bivalent radical selected from a C1-5 alkylene, C2-5 alkenylene or C2-5 alkynylene radical which may be interrupted by a heterocyclic atom; and n is between 2 and 2000; or
b. containing a repeat unit having the structure 2 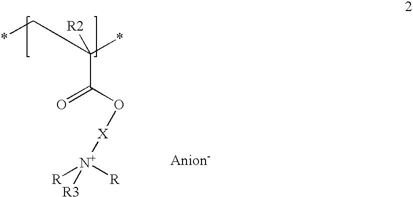
in which R, X, R2 and n are as defined above; and R3 is H, lower C1-12 alkyl, lower C2-12 alkenyl or lower C2-12 alkynyl or a substituted or unsubstituted aryl; and anion is a monovalent anion;
or mixture of repeat units of structure 1 and 2.

9. A topical composition according to claim 8, in which the mucosal surface is selected from the group consisting of ocular, respiratory, alimentary, reproductive, urinary, and exposed or injured epithelial surfaces.

10. A topical composition according to claim 8, in which the at least one polymer is dissolved in the topically acceptable vehicle.

11. A topical composition according to claim 8, in which the at least one polymer has a polydispersity index between about 1.00 and about 5.00.

12. A topical composition according to claim 8, in which the weight average molecular weight of the at least one polymer is less than about 500,000.

13. A topical composition according to claim 11, in which the weight average molecular weight of the at least one polymer is less than about 500,000.

14. A topical composition according to claim 8, in which each R is independently hydrogen or a C1-2 lower alkyl; R2 is hydrogen or a C1-2 lower alkyl; and X is a C1-2 lower alkylene radical.

15. A topical composition according to claim 8, in which each R is independently methyl; R2 is methyl; and X is an ethylene radical.

16. A method according to claim 1, in which the number average molecular weight of the at least one polymer is less than about 25,000.

17. A method according to claim 3, in which the number average molecular weight of the at least one polymer is less than about 25,000.

18. A topical composition according to claim 8, in which the number average molecular weight of the at least one polymer is less than about 25,000.

19. A topical composition according to claim 11, in which the number average molecular weight of the at least one polymer is less than about 25,000.
